(19) United States
(12) Patent Application Publication
(10) Pub. No.: US 2009/0220379 A1
(43) Pub. Date: Sep. 3, 2009

(54) ANALYZER AND MEASUREMENT RESTARTING METHOD

(75) Inventors: Yuji WAKAMIYA, Kobe-shi (JP); Hisato TAKEHARA, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/254,510

(22) Filed: Oct. 20, 2008

(30) Foreign Application Priority Data
Feb. 29, 2008 (JP) 2008-048919
Mar. 28, 2008 (JP) 2008-086191

(51) Int. Cl. G01N 33/00; G01D 11/00

(52) U.S. Cl. 422/65; 702/150

(57) ABSTRACT

An analyzer includes a measuring section for measuring samples; a transporting section for transporting a sample rack which holds sample container containing the sample to the measuring section; a motion controller for controlling the measuring section and the transporting section; an error detector for detecting an error of the analyzer; a display section; a display controller for displaying on the display section information representing handling of the sample rack present on the transporting section when the error detector detects the error; and a restart command receiver for receiving an instruction for measurement restart when the error occurs in the analyzer, wherein the motion controller controls the measuring section and the transporting section so as to selectively suction a sample required to be suctioned when the restart command receiver receives the instruction for measurement restart is disclosed. A measurement restarting method is also disclosed.

### FIELD OF THE INVENTION

The present invention relates to an analyzer in which a user can restart measurement when an error occurring in the analyzer is detected and a measurement restarting method when the error occurs.

### BACKGROUND

Analyzers for analyzing a sample collected from a living body, such as a blood-clotting analyzer and an immunological analyzer, have been used. Hospitals and medical laboratories such as inspection centers require the efficiency of inspections to rapidly return analysis results to patients from the analyzers. Accordingly, for these analyzers, transporting devices for automatically and sequentially transporting the sample to the analyzers are used.

In addition, an analyzer in which a user of the analyzer can easily recognize an error by using a light source, a speaker and a display when the error is generated in such an analyzer, for example, US Patent No. 2005036913 is proposed.

When the error is generated in the analyzer including a transporting device, the user of the analyzer is required to reset a sample rack in a predetermined position in the transporting device and restart an analysis in order to restart the stopped analysis.

However, in the technique of US Patent No. 2005036913, it is described that the user of the analyzer recognizes generation of the error of the analyzer including the transporting device and the content of the error, but a specific method for recovering the error, which can be performed by the user, is not described. Accordingly, for example, when an error requiring resetting of the sample rack in a predetermined position in the transporting device is generated, the user has to perform the recovering of the error by reading an instruction manual and thus the operation is complicated.

### SUMMARY OF THE INVENTION

The scope of the present invention is defined solely by the appended claims, and is not affected to any degree by the statements within this summary.

A first aspect of the present invention is an analyzer comprising: a measuring section for measuring samples; a transporting section for transporting a sample rack which holds sample container containing the sample to the measuring section; a motion controller for controlling the measuring section and the transporting section; an error detector for detecting an error of the analyzer; a display section; a display controller for displaying on the display section information representing handling of the sample rack present on the transporting section when the error detector detects the error; and a restart command receiver for receiving an instruction for measurement restart when the error occurs in the analyzer, wherein the motion controller controls the measuring section and the transporting section so as to selectively suction a sample required to be suctioned when the restart command receiver receives the instruction for measurement restart.

A second aspect of the present invention is a measurement restarting method comprising: transporting a sample rack to a measuring section by a transporting section for transporting the sample rack which holds sample container containing sample to the measuring section; subjecting a sample to measurement by the measuring section for performing the measurement of the sample; detecting an error of the analyzer; displaying information representing handling of the sample rack present on the transporting section when the error of the analyzer is detected; and selectively suctioning a sample required to be suctioned when an instruction for measurement restart is received after detection of the error.

### BRIEF DESCRIPTION OF THE DRAWINGS

FIG. 1 is an example of an overall configuration diagram of an analyzer according to a first embodiment;

FIG. 2 is a hardware configuration diagram of a control device illustrated in FIG. 1;

FIG. 3 is a hardware configuration diagram of a measurement control section illustrated in FIG. 1;

FIG. 4 is a perspective view of a sample rack holding test tubes;

FIG. 5 is a hardware configuration diagram of a transporting section;

FIG. 6 is an example of a flowchart illustrating a main process which is performed by the measurement control section (first embodiment);

FIG. 7 is an example of a flowchart illustrating a main process which is performed by the control device (first embodiment);

FIG. 8 is an example of a flowchart illustrating an error processing which is performed by the analyzer (first embodiment);

FIG. 9 is a diagram illustrating an example of a screen where a user registers measurement information;

FIG. 10 is an example of a sample rack information management table which is managed by the measurement control section;

FIG. 11 is an example of data inquiring about measurement information from the control device to the measurement control section;

FIG. 12A is an example of measurement information transmitted to the measurement control section from the control device;

FIG. 12B is an example of measurement information transmitted to the measurement control section from the control device;

FIG. 13A is an example of data transmitted to the control device from the measurement control section upon suction completion of a sample;

FIG. 13B is an example of information representing whether there are orders of measuring items;

FIG. 14A is an example of a database managing the measurement information stored in a hard disk 313;

FIG. 14B is an example of measuring item suction information among the measurement information stored in the hard disk 313;

FIG. 14C is an example of measuring item suction information among the measurement information stored in the hard disk 313;

FIG. 15 is an example of data transmitted to the control device from the measurement control section when an error is generated in the analyzer;

FIG. 16 is an example of data transmitted to the control device from the measurement control section when a sample rack other than the sample racks stored in the sample rack information management table is reset after the error is generated in the analyzer;

FIG. 17 is an example of the transporting section when one sample rack is present on a transporting line;

FIG. 18 is an example of a help screen which is displayed when one sample rack is present on the transporting line (first embodiment);

FIG. 19 is an example of the transporting section when two sample racks are present on the transporting line;

FIG. 20 is an example of a help screen which is displayed when two sample racks are present on the transporting line (first embodiment);

FIG. 21 is an example of a flowchart illustrating a main process which is performed by the control device (second embodiment);

FIG. 22 is an example of a flowchart illustrating a main process which is performed by the control device (second embodiment);

FIG. 23 is example of a flowchart illustrating an error processing which is performed by the analyzer (second embodiment);

FIG. 24 is an example of data transmitted to the control device from the measurement control section when an error is generated in the analyzer (second embodiment);

FIG. 25 is an example of a help screen which is displayed when an error is generated in the transporting section (second embodiment); and

FIG. 26 is an example of the sample racks in the transporting section when the error is generated in the transporting section (second embodiment).

### DETAILED DESCRIPTION OF THE PREFERRED EMBODIMENTS

The preferred embodiments of the present invention will be described hereinafter with reference to the drawings.

### First Embodiment

Hereinafter, a first embodiment of the invention will be described in detail with reference to the drawings.

[Overall Configuration of Device]

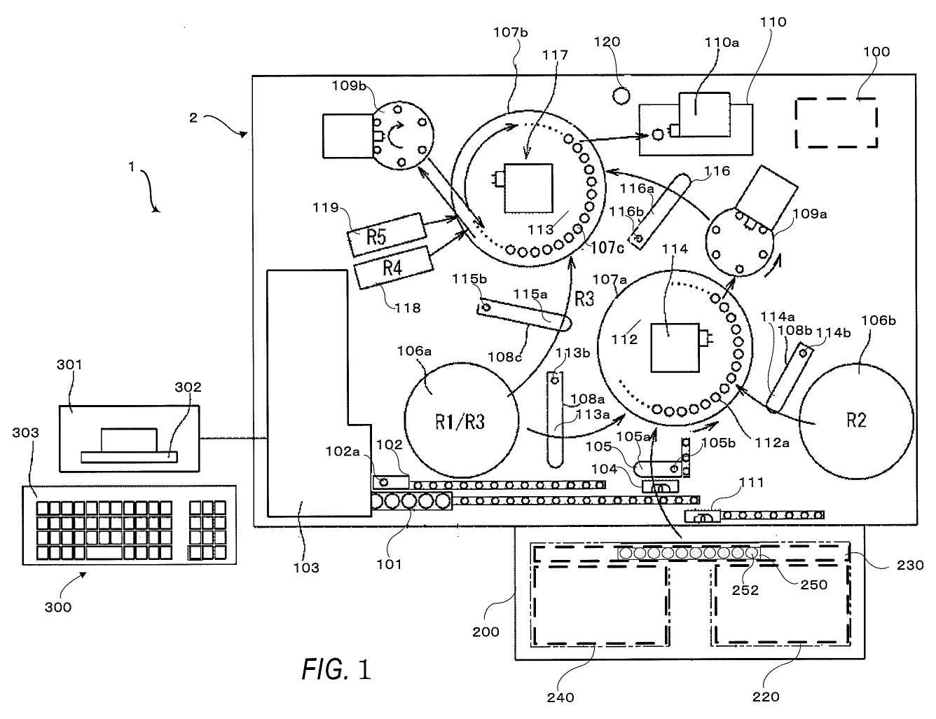

FIG. 1 is a plan explanatory diagram illustrating the overall configuration of an immunological analyzer (sample analyzer) according to an embodiment of the invention.

The immunological analyzer 1 is a device for inspecting various measuring items such as hepatitis B, hepatitis C, tumor marker, and thyroid hormone by using a sample (specimen) such as blood. As illustrated in FIG. 1, the immunological analyzer 1 is configured by a measuring unit (measuring section) 2 including a plurality of mechanisms (components) and a control device 300 as a data processing unit which is electrically connected to the measuring unit 2.

With the immunological analyzer 1, capture antibodies (reagent R1) are bound to antigens included in the sample such as blood as a measuring target to form complexes of antigen-capture antibody. Next, magnetic particles (reagent R2) are bound to the complexes of antigen-capture antibody to form complexes of antigen-capture body-magnetic particle. Then, the complexes of antigen-capture body-magnetic particle are drawn to a magnet of a first BF (Bound Free) separating section 109a to remove the unreacted (Free) capture antibodies. In addition, labeled antibodies (reagent R3) are bound to the complexes of antigen-capture body-magnetic particle to form complexes of labeled antibody-antigen-capture body-magnetic particle. After that, the complexes of labeled antibody-antigen-capture body-magnetic particle are drawn to a magnet of a second BF separating section 109b to remove the unreacted (Free) labeled antibodies. Further, luminescent substrates (reagent R5) emitting light in the course of the reaction with the labeled antibodies are added and then an amount of luminescence generated by the reaction of the labeled antibodies with the luminescent substrates is measured. Through such a course, the antigens included in the sample bound to the labeled antibodies are quantitatively measured.

[Configuration of Measuring Unit]

The measuring unit 2 has a measurement control section 100, a transporting section 200, a bar-code reader 111, an urgent sample transporting section 101, a tip transporting section 102, a pipette tip supply device 103, a tip removing section 104, a suction section 105, reagent mounting sections 106a and 106b, a first reaction section 107a, a second reaction section 107b, reagent dispensing arms 108a, 108b, and 108c, the first BF separating section 109a, the second BF separating section 109b, and a detecting section 110.

The mechanisms of the measuring unit 2 can properly employ known configurations. However, hereinafter, the configurations thereof will be simply described with reference to FIG. 1.

The measurement control section 100 has a CPU, a RAM, and a ROM, controls the transporting section 200, the urgent sample transporting section 101, the detecting section 110 and the suction section 105, and transmits a detection result received from the detecting section 110 to the control device 300 through a communication interface 351.

The transporting section 200 has a right tank section 220 for setting a sample rack 250 holding a plurality of test tubes 252, a transporting line 230 for transporting the sample rack 250 fed from the right tack section 220 to a position where it is sent to a left tank section 240, and the left tack section 240 for storing the sample rack 250 sent from the transporting line 230.

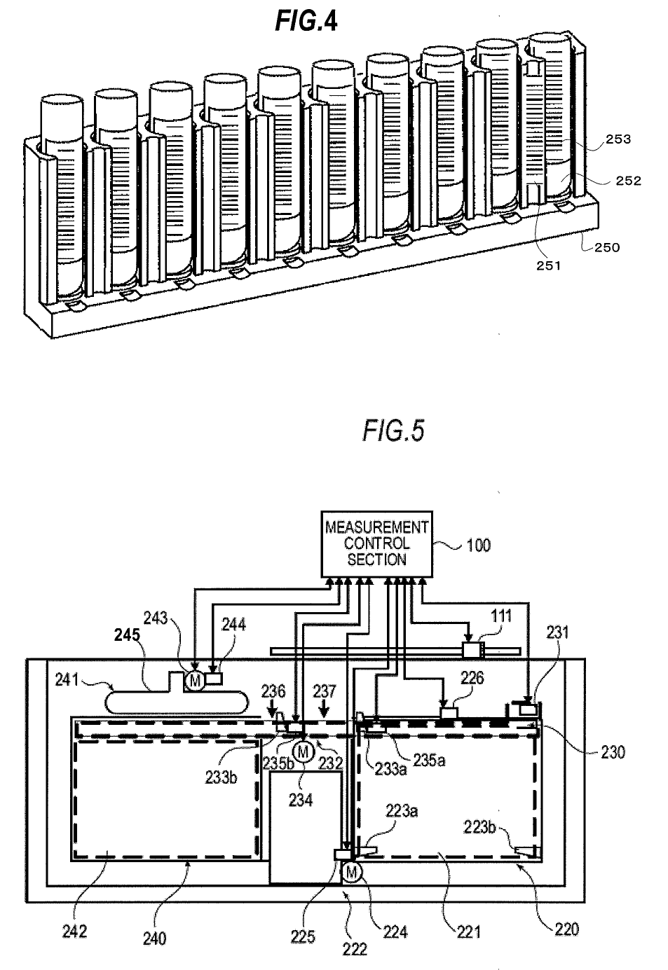

FIG. 4 is a perspective view of the sample rack 250 holding the plurality of test tubes 252 containing samples. A sample rack bar-code 251 for identifying the sample rack 250 is adhered to the sample rack 250 and is read by the bar-code reader 111. As the sample rack bar-code 251, a bar-code such as CODE 128, CO39, NW-7 or the like can be used.

A test tube bar-code 253 for identifying a sample is adhered to a test tube 252 and is read by the bar-code reader 111. As the test tube bar-code 253, a bar-code such as CODE 128, CO39, NW-7 or the like can be used.

Returning to FIG. 1, the bar-code reader 111 is configured so as to read the sample rack bar-code 251 of the sample rack 250 fed to the transporting line 230 from the right tank section 220 of the transporting section 200 and the test tube bar-code 253 of the test tube 252 held in the sample rack 250.

FIG. 5 is a diagram schematically illustrating the transporting section 200 and the bar-code reader 111. The transporting section mainly has the right tank section 220, the transporting line 230 and the left tank section 240.

The right tank section 220 has a sample rack setting section 221, a sample rack presence/absence sensor 226 and a sample rack feeding mechanism section 222.

For recovering errors of the measuring and analyzing devices, a user sets the sample rack 250 holding the test tube 252 in the sample rack setting section 221 such that the sample rack bar-code 251 is opposed to the bar-code reader 111.

The sample rack presence/absence sensor 226 is provided to detect that the sample rack is set in the sample rack setting section 221.

The sample rack feeding mechanism section 222 has feeding levers 223a and 223b, a motor 224 and a sensor 225. The feeding levers 223a and 223b are driven by the motor 224 to feed the sample rack 250 on the sample rack setting section 221 to the transporting line 230. Further, the sensor 225 is provided to detect that the feeding levers 223a and 223b return to the origin positions thereof.

Next, the transporting line 230 has a sample rack arrival sensor 231, a sample rack transverse sending mechanism 232, a suction position 236 and a measurement information inquiry position 237 and can accommodate two sample racks 250.

The sample rack arrival sensor 231 is provided to detect that the sample rack 250 in the sample rack setting section 221 is normally fed to the transporting line 230 by the sample rack feeding mechanism section 222.

The sample rack transverse sending mechanism 232 has transverse sending levers 233a and 233b, a motor 234, sensors 235a and 235b. The transverse sending levers 233a and 233b are connected to each other and are synchronized by the motor 234 so as to be driven. When the levers 233a and 233b are driven, they are caught on a bottom section of the sample rack 250 and then the sample rack 250 is transversely sent by one pitch (by a distance corresponding to one test tube) in a direction of the left tank section 220. The sensors 235a and 235b are provided to detect that the transverse sending levers 233 normally move and that the sample rack 250 does not move when the motor 234 stops.

The sample rack transverse sending mechanism 232 repeatedly performs the transverse movement of the sample rack 250 until the sample rack 250 reaches the position where the sample rack on the transporting line 230 is sent.

The suction position 236 represents that the sample is suctioned from the test tube 252 arriving at this position. When the test tube 252 requiring a suction operation for measurement arrives at the suction position 236, the measurement control section 100 controls the suction section 105 to suction the sample from the test tube 252 at the suction position 236.

From the measurement control section 100, an inquiry is made to the control device 300 about measurement information of the test tube 252 arriving at the measurement information inquiry position 237.

Next, after the sample rack 250 fed to the transporting line 230 is transversely sent once, the bar-code reader 111 reads the sample rack bar-code 251 and the test tube bar-codes 253 of all of the test tubes 252 held in the sample rack 250.

The left tank section 240 has a sample rack sending mechanism section 241 and a sample rack discharging section 242.

The sample rack sending mechanism section 241 has a sending lever 245, a motor 243 and a sensor 244. When the sample rack 250 arrives at a left end of the transporting line 230, the sending lever 245 is driven by the motor 243 to send the sample rack 250 to the sample rack discharging section 242. The sensor 244 is provided to detect that the sending lever 245 normally returns to the origin position thereof.

The sample rack discharging section 242 stores the sample rack 250 sent by the sample rack sending mechanism section 241 and the user removes the sample rack 250 in which the sample has been suctioned from the sample rack discharging section 242.

Returning to FIG. 1, the urgent sample transporting section 101 is configured so as to transport the test tube 252 containing an urgent sample requiring to be inspected by entering into the sample transported by the transporting section 200 to a suction position for the urgent sample.

The pipette tip supply device 103 has a function of supplying a put pipette tip to a tip mounting section 102a of the tip transporting section 102 one by one.

The tip removing section 104 is provided to remove the pipette tip mounted on the suction section 105 to be described later.

The suction section 105 has a function of dispensing the sample in the test tube 252 transported to the suction position by the transporting section 200 into a cuvette (not shown) held in a holding section 112a of a rotation table section 112 of the first reaction section 107a to be described later. The suction section 105 is configured so as to rotate an arm section 105a around a shaft 105b in a front-and-back direction and to operate the arm section 105a in an up-and-down direction. Further, a nozzle section for suctioning and ejecting the sample is provided at a tip end of the arm section 105a. A tip end of the nozzle section is mounted with the pipette tip transported by the tip transporting section 102.

On the reagent mounting section 106a, a reagent container containing the reagent R1 including the capture antibodies and a reagent container containing the reagent R3 including the labeled antibodies are mounted.

On the reagent mounting section 106b, reagent container containing the reagent R2 including the magnetic particles is mounted.

The first reaction section 107a is provided to rotate and move by a predetermined angle at predetermined intervals (in this embodiment, 18 seconds) the cuvette held in the holding section 112a of the rotation table section 112 and to stir the reagents R1 and R2 and the liquid in the cuvette. That is, the first reaction section 107a is provided to react the reagent R2 having the magnetic particles with the antigens in the sample in the cuvette. The first reaction section 107a is configured by the rotation table section 112 for transporting the cuvette containing the reagents R1 and R2 and the sample in a rotation direction and a container transporting section 114 for stirring the reagents R1 and R2 and the sample in the cuvette and transporting the cuvette containing the stirred reagents R1 and R2 and sample to the first BF separating section 109a to be described later.

The container transporting section 114 is rotatably mounted at the center of the rotation table section 112. The container transporting section 114 has a function of grasping the cuvette held in the holding section 112a of the rotation table section 112 and stirring the specimen in the cuvette. In addition, the container transporting section 114 also has a function of transporting the cuvette containing the specimen obtained by stirring and incubating the reagents R1 and R2 and the sample to the first BF separating section 109a.

The second reaction section 107b has the same configuration as the first reaction section 107a and is provided to rotate and move by a predetermined angle at predetermined intervals (in this embodiment, 18 seconds) the cuvette held in a holding section 107c of a rotation table section 113 and to stir the reagents R1, R2, R3 and R5 and the sample in the cuvette. That is, the second reaction section 107b is provided to react the reagent R3 having the labeled antibodies with the antigens in the sample and to react the reagent R5 having the luminescent substrates with the labeled antibodies of the reagent R3 in the cuvette. The second reaction section 107b is configured by the rotation table section 113 for transporting the cuvette containing the reagents R1, R2, R4 and R5 and the sample in a rotation direction and a container transporting section 117 for stirring the reagents R1, R2, R3 and R5 and the sample in the cuvette and transporting the cuvette containing the stirred sample and the like to the second BF separating section 109b to be described later. The container transporting section 117 has a function of transporting the cuvette processed by the second BF separating section 109b to the holding section 107c of the rotation table section 113 again.

The reagent dispensing arm 108a has a function of suctioning the reagent R1 in the reagent container mounted on the reagent mounting section 106a and dispensing the suctioned reagent R1 into the cuvette of the first reaction section 107a. The reagent dispensing arm 108a is configured so as to rotate an arm section 113a around a shaft 113b and move it in an up-and down direction. Further, a nozzle for suctioning and ejecting the reagent R1 in the reagent container is attached to a tip end of the arm section 113a.

The reagent dispensing arm 108b has a function of dispensing the reagent R2 in the reagent container mounted on the reagent mounting section 106b into the cuvette into which the sample and the reagent R1 of the first reaction section 107a are dispensed. The reagent dispensing arm 108b is configured so as to rotate an arm section 114a around a shaft 114b and move it in an up-and-down direction. Further, a nozzle for suctioning and ejecting the reagent R2 in the reagent container is attached to a tip end of the arm section 114a.

The reagent dispensing arm 108c has a function of suctioning the reagent R3 in the reagent container mounted on the reagent mounting section 106a and dispensing the suctioned reagent R3 into the cuvette into which the reagents R1 and R2 and the sample of the second reaction section 107b are dispensed. The reagent dispensing arm 108c is configured so as to rotate an arm section 115a around a shaft 115b and move it in an up-and-down direction. Further, a nozzle for suctioning and ejecting the reagent R3 in the reagent container is attached to a tip end of the arm section 115a.

The first BF separating section 109a is provided to separate the unreacted reagent R1 (unnecessary components) and the magnetic particles from the specimen in the cuvette transported by the container transporting section 114 of the first reaction section 107a.

The cuvette of the first BF separating section 109a from which the unreacted reagent R1 and the like are separated is transported to the holding section 107c of the rotation table 113 of the second reaction section 107b by a transporting mechanism 116. The transporting mechanism 116 is configured so as to rotate an arm section 116a having a cuvette grasping section (not shown) at a tip end thereof around a shaft 116b and move it in an up-and-down direction.

The second BF separating section 109b has the same configuration as the first BF separating section 109a and is provided to separate the unreacted reagent R3 (unnecessary components) and the magnetic particles from the specimen in the cuvette transported by the container transporting section 117 of the second reaction section 107b.

Respectively, a reagent R4 supply section 118 and a reagent R5 supply section 119 are provided to supply the reagent R4 and the reagent R5 to the cuvette held in the holding section 107c of the rotation table section 113 of the second reaction section 107b.

The detecting section 110 is provided to acquire the light generated in the course of the reaction of the luminescent substrates with the labeled antibodies bound to the antigens of the sample subjected to a predetermined process by a photomultiplier tube to thereby measure an amount of the antigens included in the sample. The detecting section 110 has a transporting mechanism section 110a for transporting the cuvette held in the holding section 107c of the rotation table section 113 of the second reaction section 107b to the detecting section 110.

The used cuvette, in which the specimen subjected to the measurement is suctioned, is discarded into a dust box (not shown) disposed under the immunological analyzer 1 through a waste hole 120.

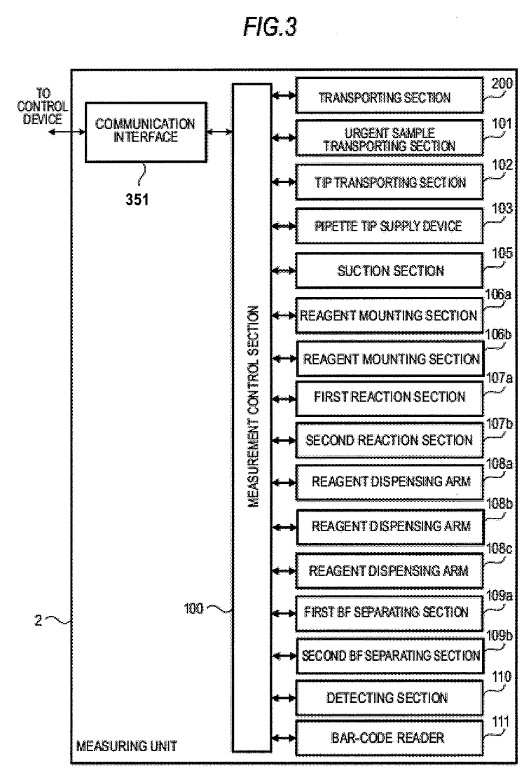

As illustrated in FIG. 3, in the measuring unit 2, the measurement control section 100 controls the mechanisms. In addition, the measuring control section receives measurement information from the control device 300, transmits a measurement result to the control device 300 and notifies the control device 300 of an error through the communication interface 351 using Ethernet (registered trade name).

[Control Device]

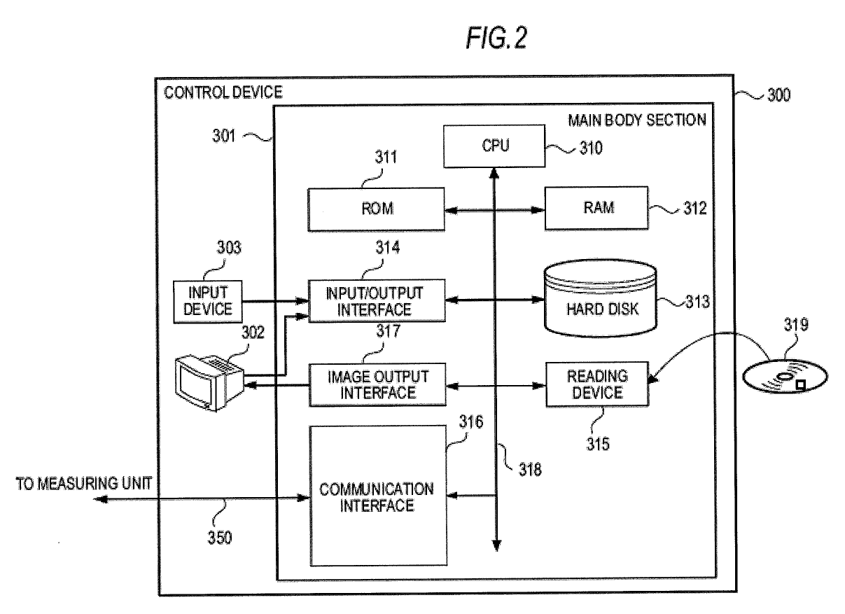

FIG. 2 illustrates a block diagram of the control device 300. As illustrated in FIG. 2, the control device 300 is a computer mainly configured by a main body section 301, a display 302 and an input device 303.

The main body section 301 is mainly configured by a CPU 310, a ROM 311, a RAM 312, a hard disk 313, an input/output interface 314, a reading device 315, a communication interface 316 and an image output interface 317. The CPU 310, ROM 311, RAM 312, hard disk 313, input/output interface 314, reading device 315, communication interface 316 and image output interface 317 are connected to each other by a bus 318 such that data communication can be mutually performed.

The CPU 310 can execute computer programs stored in the ROM 311 and the hard disk 313 and a computer program loaded to the RAM 312. By executing an application program on the CUP 310, the functional blocks to be described later are realized and the computer functions as the control device 300.

The ROM 311 includes a mask ROM, a PROM, an EPROM and an EEPROM and a computer program to be executed on the CPU 310 and data to be used for the computer program are recorded therein.

The RAM 312 includes a SRAM and a DRAM. The RAM 312 is used to read computer programs recorded in the ROM 311 and the hard disk 313. Moreover, the RAM 312 is used as a work area of the CPU 310 when the computer programs are executed.

On the hard disk 313, various computer programs for being executed on the CPU 310, such as an operating system and an application program, and data to be used for the computer programs are installed.

The reading device 315 includes a flexible disk drive, a CD-ROM drive, and a DVD-ROM drive to read a computer program or data recorded in a portable recording medium 319.

The input/output interface 314 includes, for example, a serial interface such as USB, IEEE1394 and RS-232C, a parallel interface such as SCSI, IDE, and IEEE1284, and an analog interface including a D/A converter and an A/D converter. The input/output interface 314 is connected to the input device 303 including a keyboard, a mouse and a handy bar-code reader. An operator can input data to the main body 301 by using the input device 303.

The communication interface 316 is, for example, an Ethernet (registered trade name) interface. Through the communication interface 316, the control device 300 can send and receive data to and from the measurement control section 100 connected via the network 350 by using a predetermined protocol.

The image output interface 317 is connected to the display 302 including LCD and CRT to output a picture signal corresponding to image data given from the CPU 310 to the display 302.

The display 302 displays an image (screen) in accordance with the input picture signal.

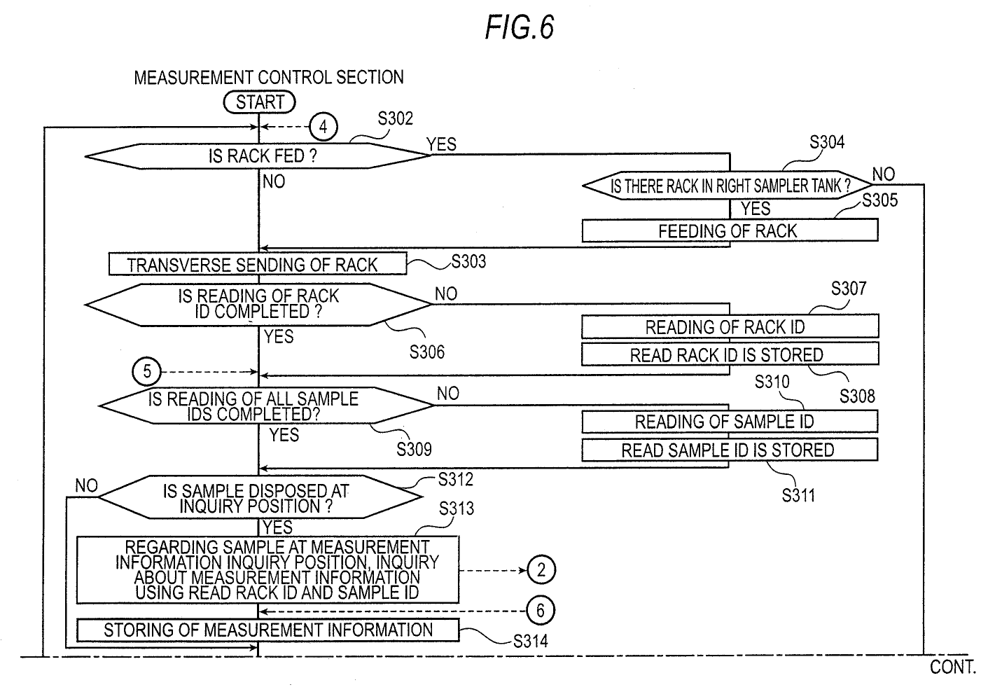

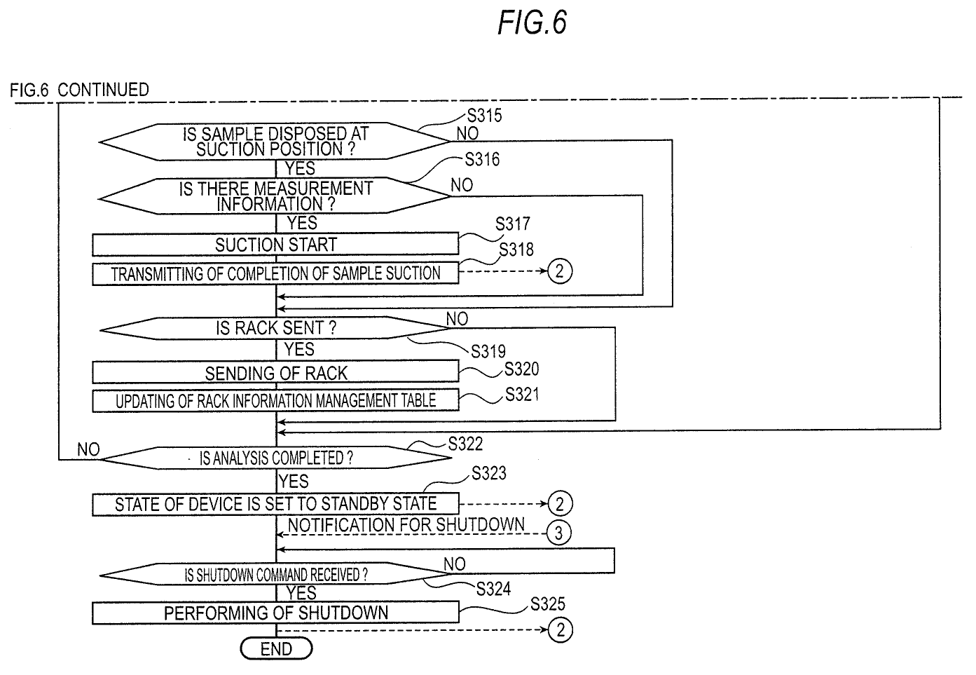

Hereinafter, using FIGS. 6 to 20, a sample measuring process according to the first embodiment will be described.

[Overall Process]

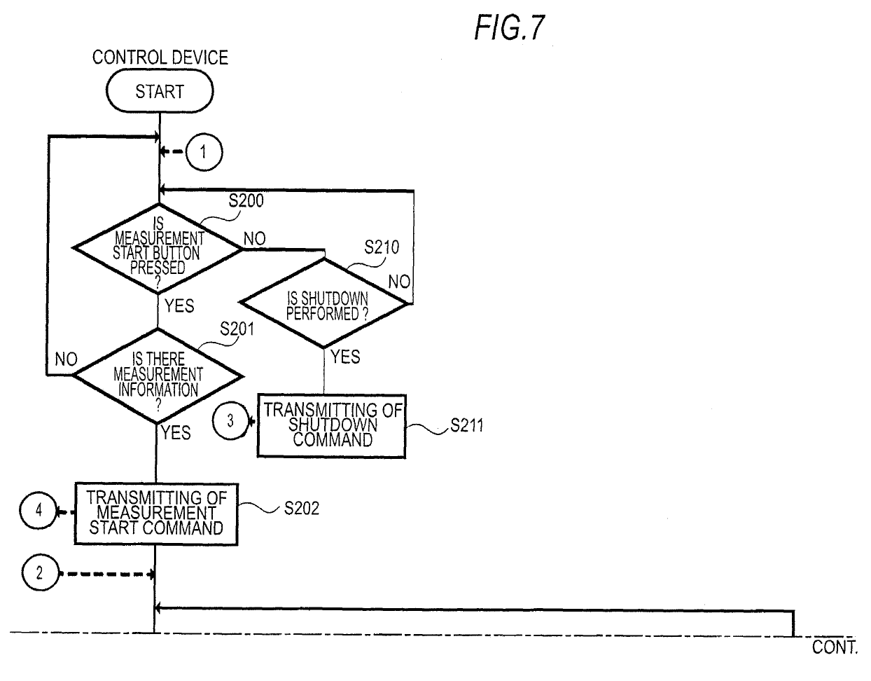

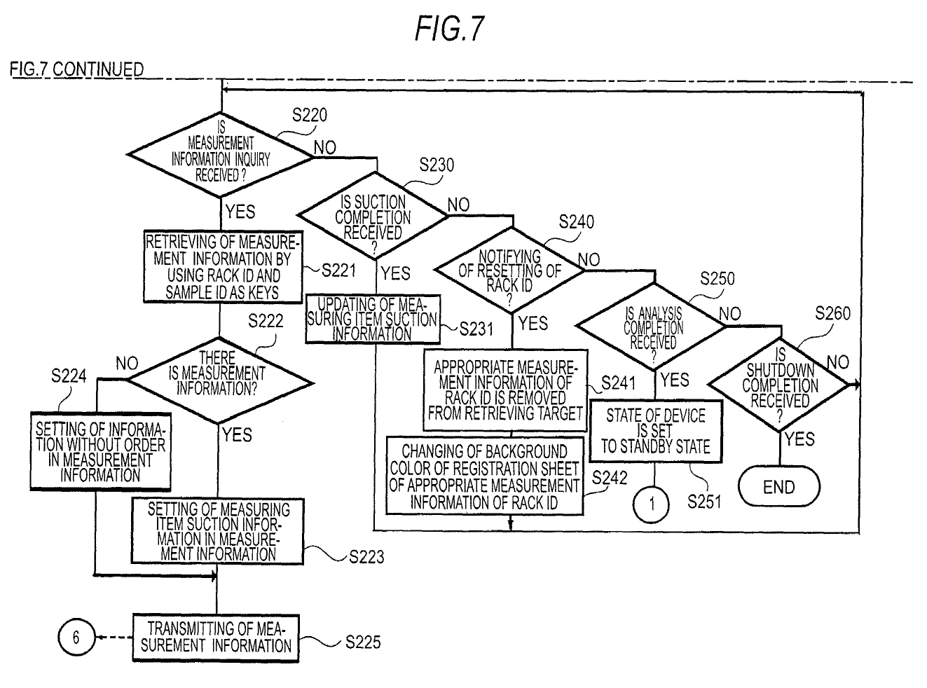

FIGS. 6 and 7 are flowcharts illustrating the processes when the measurement is normally performed in the immunological analyzer 1.

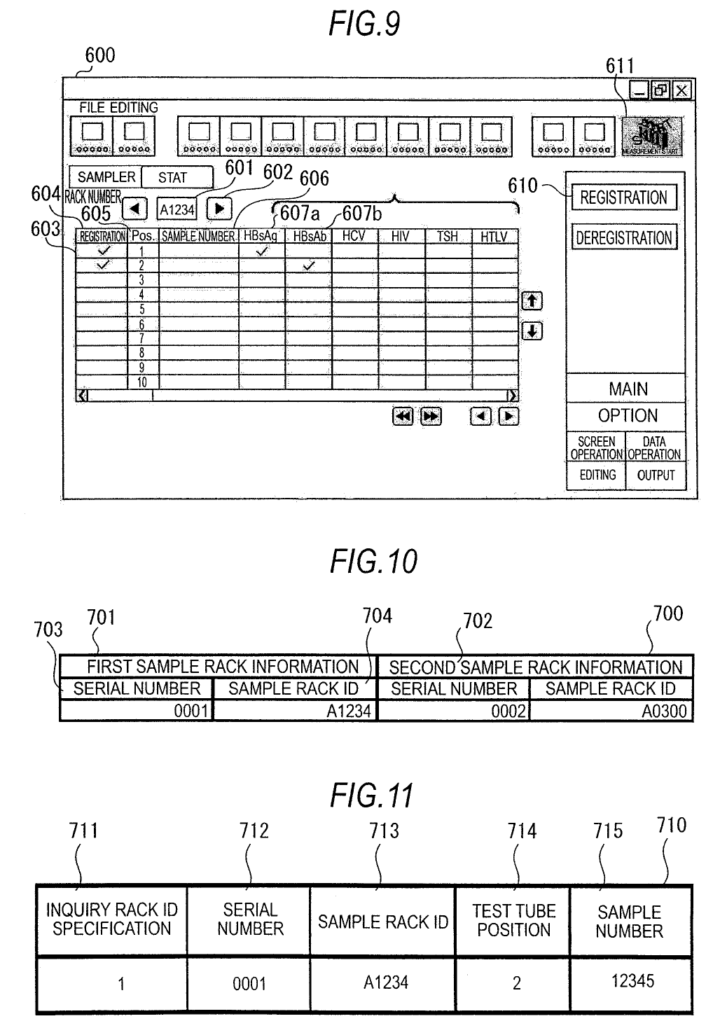

In Step S200 illustrate in FIG. 7, when the user presses a measurement start button (reference numeral 611 of FIG. 9 to be described later) shown on the display 302 of the control device 300 (Yes in Step S200), determining whether the measurement information is input by the user is performed (Step S201). When the measurement information is input by the user (Yes in. Step S201), the measurement control section 100 is notified of measurement start (Step S202). When the user presses the measurement start button 611 but the measurement information is not input (No in Step S201), the process returns to Step S200.

Moving to FIG. 6, when the notification for measurement start is received, the measurement control section 100 determines whether the sample rack 250 is fed to the transporting line 230 (Step S302). When it is determined that the sample rack 250 is to be fed (Yes in Step S302), determining whether the sample rack 250 is present in the sample rack setting section 221 is performed on the basis of the output of the sensor 226 (Step S304). When the sample rack 250 is present in the sample rack setting section 221 (Yes in Step S304), the sample rack 250 is fed to the transporting line 230 by the sample rack feeding mechanism section 222 (Step S305).

In Step S305, when the sample rack arrival sensor 231 cannot detect that the sample rack 250 is fed even after the sample rack feeding mechanism section 222 is driven and a predetermined period of time passes, the measurement control section 100 performs a process upon generation of an error to be described later.

Further, when returning of the sample rack feeding mechanism section 222 to the origin position thereof cannot be detected on the basis of the output of the sensor 225 even after the feeding of the sample rack 250 to the transporting line 230 is completed by the sample rack feeding mechanism section 222 and a predetermined period of time passes, the measurement control section 100 performs the process upon generation of the error to be described later.

When the sample rack 250 is not present in the sample rack setting section 221 (No in Step S304), the process proceeds to Step S322.

When it is determined that the sample rack 250 is not to be fed, that is, when a new sample rack 250 is fed to the transporting line 230 and it is determined that the new sample rack interferes with the sample rack 250 already present on the transporting line (No in Step S302), the measurement control section 100 does not perform the feeding of the sample rack 250 and the process proceeds to Step S303.

Next, the sample rack 250 on the transporting line 230 is transversely sent once (Step S303). In this Step S303, when it is detected on the basis of the output of the sensors 235a and 235b that the transverse sending levers 233 are not normally operated, or that the sample rack 250 is moved upon stopping of the motor 234, the measurement control section 100 performs the process upon generation of the error to be described later.

Next, the measurement control section 100 checks whether the information of the sample rack bar-code 251 has been acquired by the bar-code reader 111 (Step S306). When the information of the sample rack bar-code 251 has not yet been acquired (No in Step S306), the information of the sample rack bar-code 251 is acquired by the bar-code reader 111 (Step S307) and the acquired information of the sample rack bar-code 251 is stored (Step S308).

When the information of the sample rack bar-code 251 has been acquired (Yes in Step S306) or when the process proceeds to Step S309 after Step S308 and there is the test tube bar-code 253 of the test tube 252 held in the sample rack 250, which has not yet been acquired (No in Step S309), the test tube bar-code 253 which has not yet been acquired is acquired by the bar-code reader 111 (Step S310) and the acquired information is stored (Step S311).

FIG. 10 is a diagram schematically illustrating a sample rack information management table 700 which is stored in the measurement control section 100 in the above-described Step S308.

The sample rack information management table 700 has first sample rack information 701 and second sample rack information 702. The first sample rack information 701 represents the information of the sample rack 250 initially fed to the transporting line 230 among the sample racks 250 present on the transporting line 230. The second sample rack information 702 represents the information of the sample rack 250 finally fed to the transporting line 230 among the sample racks 250 present on the transporting line 230.

Each of the first sample rack information 701 and the second sample rack information 702 has a serial number 703 and a sample rack ID 704. The serial number 703 is a serial number which is applied to the sample rack 250 by the measurement control section 100 every time the sample rack 250 is fed to the transporting line 230 after the measurement control section 100 is turned on. Unique numbers are applied until the measuring unit 2 is shutdown. The sample rack ID 704 represents the information of the sample rack bar-code 251 acquired by the bar-code reader 111.

For example, the sample rack information management table 700 illustrated in FIG. 10 represents that the number of the sample racks 250 present on the transporting line 230 is currently two, that the serial number 703 of the sample rack 250 firstly fed to the transporting line 230 is 0001 and that the information of the sample rack bar-code 251 of the firstly fed sample rack, acquired by the bar-code reader 111, is A1234. Further, the sample rack information management table represents that the serial number 703 of the sample rack 250 secondly fed to the transporting line 230 is 0002 and that the information of the sample rack bar-code 251 of the secondly fed sample rack, acquired by the bar-code reader 111, is A0300.

Returning to FIG. 6, when the test tube 252 is present at the measurement information inquiry position 237 (Yes in Step S312), an inquiry is made to the control device 300 about the measurement information of the test tube 252 present at the measurement information inquiry position 237 (Step S313). When the test tube 252 is not present at the measurement information inquiry position 237 (No in Step S312), the process proceeds to Step S315.

Returning to FIG. 7, when measurement information inquiry data 710 illustrated in FIG. 11 is received (Yes in Step S220), the control device 300 retrieves the measurement information from the measurement information stored in a predetermined area of the hard disk 313 illustrated in FIG. 2 by using the information of the sample rack bar-code 251, the information of the test tube bar-code 253 and the test tube position in the sample rack 250 holding the test tube 252 as keys (Step S221).

FIG. 11 is a diagram schematically illustrating the measurement information inquiry data 710 transmitted to the control device 300 from the measurement control section 100.

The measurement information inquiry data 710 has inquiry rack ID specifying information 711, a serial number 712, a sample rack ID 713, a test tube position 714 and a sample number 715.

The inquiry rack ID specifying information 711 is information specifying which one of the serial number 712 and the sample rack ID 713 is used as a key for the sample rack when the control device 300 retrieves the measurement information about the inquired sample from the measurement information stored in the hard disk 313. When 0 is set, the serial number 712 is used as a key, and when 1 is set, the sample rack ID 713 is used as a key to retrieve the measurement information. The serial number 712 is information representing a serial number applied to the sample rack 250 by the measurement control section 100 when the sample rack 250 is fed to the transporting line 230. The sample rack ID 713 represents the information of the sample rack bar-code 251 acquired by the bar-code reader 111. The test tube position 714 is information representing the test tube position of the test tube 252 in the sample rack 250. The sample number 715 represents information of the test tube bar-code 253 acquired by the bar-code reader 111.

For example, the measurement information inquiry data 710 illustrated in FIG. 11 represents that the sample rack ID 713 is A1234, that the sample number 715 is 12345 and that the measurement information inquiry data 710 is data of the sample held in a second position of the sample rack 250.

FIG. 9 is an example of a measurement information registering screen where the user inputs the measurement information.

A measurement information registering screen 600 is displayed on the display 302 of the control device 300 and mainly has a sample rack ID input box 601, a page switching button 602, a measurement information input sheet 603, a registration button 610 and a measurement start button 611.

The sample rack ID input box 601 is a box to which the information of the sample rack bar-code 251 is input and the information is input by a handy bar-code reader, a keyboard or the like.

When the measurement information of the plurality of sample racks 250 is input, the page switching button 602 is pressed to display the measurement information input sheet 603 for inputting the measurement information of the next sample rack 250.

The measurement information input sheet 603 has a registration state display box 604, a test tube position 605, a sample number input box 606 and a measuring item selecting box 607. The registration state display box 604 is information representing whether the input measurement information has been registered and represents that orders for the checked samples have been registered. The sample number input box is a box to which the information of the test tube bar code 253 is input and the information is input by a handy bar-code reader, a keyboard or the like. The measuring item selecting box 607 is a box in which a measuring item to be subjected to the measurement can be selected and the measuring item to be subjected to the measurement is selected by a mouse.

By the registration button 610, the measurement information displayed on the measurement information input sheet 603 is registered and the measurement information is stored in the hard disk 313.

The measurement start button 611 notifies the measuring unit 2 of a measurement start instruction on the basis of the measurement information input to the measurement information input sheet 603 so as to start the measurement.

For example, the measurement information registering screen 600 illustrated in FIG. 9 shows that the measurement information in which regarding the sample, of which the sample rack bar-code 251 is A1234 and the information of the test tube bar-code 253 at a first test tube mounting position of the sample rack 250 is 12345, HBsAg of an item 607a is measured, and regarding the sample, of which the information of the test tube bar-code 253 at a second test tube mounting position is ABCDE, HBsAb of an item 607b is measured is stored in the hard disk 313 of the control device 300.

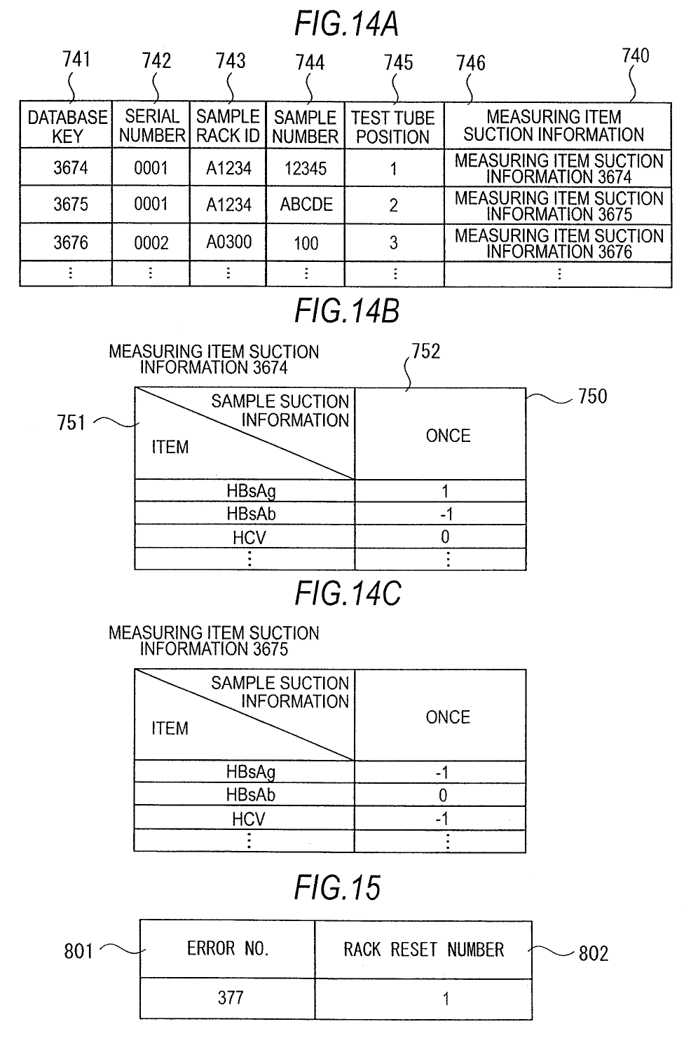

FIG. 14A is a diagram schematically illustrating a measurement information management database 740 for managing the measurement information stored in the hard disk 313.

The measurement information management database 740 mainly has a database key 741, a serial number 742, a sample rack ID 743, a sample number 744, a test tube position 745 and measuring item suction information 746.

The database key 741 represents the information for extracting the information of the retrieving target from the measurement information management database 740. The serial number 742 is information representing the serial number which is applied to the sample rack 250 put on the transporting line 230 by the measurement control section 100. The sample rack ID 743 is information representing the value input to the sample rack number input box 601 of the measurement information registering screen 600 illustrated in FIG. 9. The sample number 744 is information representing the value input to the sample number input box 606 of the measurement information registering screen 600 and corresponds to the test tube position 605 of the measurement information input sheet 603. The test tube position 745 is information representing the position of the test tube 252 corresponding to the measurement in the sample rack 250. The measuring item suction information 746 is information representing whether there are the orders of the measuring items and includes the information illustrated in FIGS. 14B and 14C.

The measuring item suction information illustrated in FIG. 14B includes a measuring item 751 and suction information 752. The measuring item 751 is information representing the measuring items which can be subjected to the measurement by the analyzer and corresponds to the measuring item selecting box 607 of the measurement information input sheet 603. The suction information 752 is information representing whether the measuring items are registered as the items to be subjected to the measurement or have been already suctioned for the measurement. 0 represents that the order for the measurement has been registered and the sample has not yet been suctioned. 1 represents that the order for the measurement has been registered and the suctioning has been completed. −1 represents that there is no order. For example, measuring item suction information 750 illustrated in FIG. 14B represents that the order of the measuring item HBsAg has been registered and the suctioning of the sample has been completed, that the order of the measuring item HBsAb has not been registered, and that the order of the measuring item HCV has been registered and the suctioning of the sample has not yet been performed.

Accordingly, the measurement information management database 740 illustrated in FIG. 14A represents that the database key 741 at an area 3674 has the serial number 742 of 0001 and the sample rack ID 743 of A1234, that the measuring items of HbsAg and HCV in the sample which is set at a first test tube position in the sample rack 250 and has the sample number 744 of 12345 are registered as orders, that the suctioning of the sample regarding the measuring item HBsAg has been completed and that the suctioning of the sample regarding the measuring item HCV has not yet been completed.

Further, the measurement information management database represents that the database key 741 at an area 3675 has the serial number 742 of 0001 and the sample rack ID number 743 of A1234, that the measuring item HbsAb in the sample which is set at a second test tube position in the sample rack 250 and has the sample number 744 of ABCDE is registered as an order and that the suctioning of the sample regarding the measuring item HbsAb has not yet been completed.

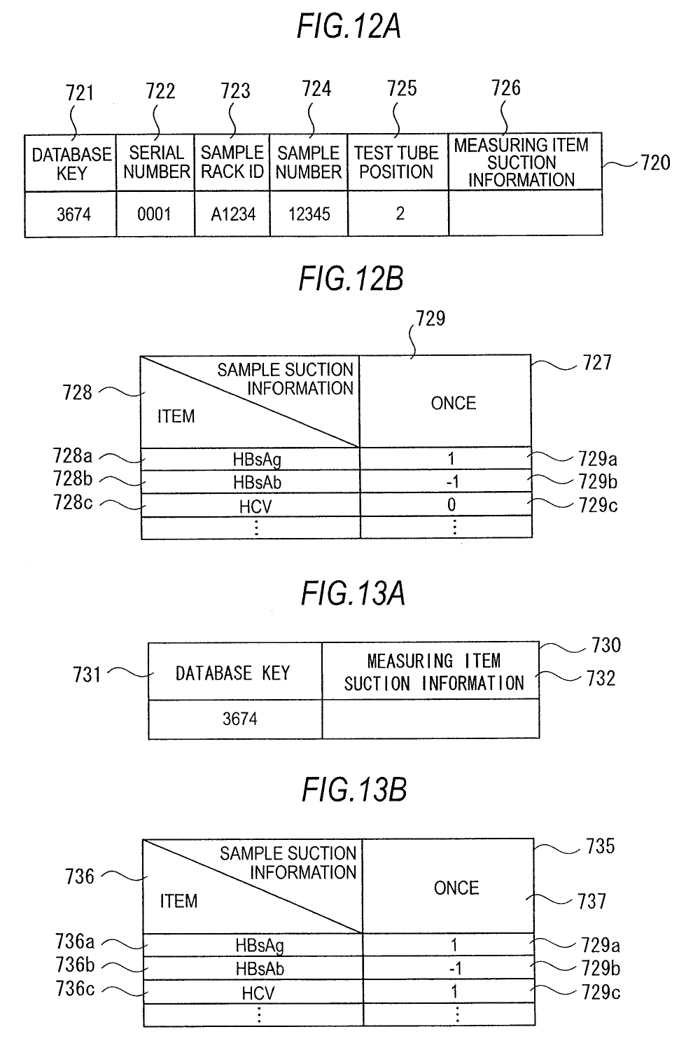

Returning to FIG. 7, when there is the appropriate measurement information after the retrieval of the measurement information (Yes in Step S222), the database key 741 and the measuring item suction information 746 are added to the measurement information inquiry data 710 (Step S223) and the measurement control section 100 is notified of the order information illustrated in FIG. 12B (Step S225). When there is not the appropriate measurement information after the retrieval of the measurement information (No in Step S222), information without an order is added to the measurement information inquiry data 710 and the measurement control section 100 is notified (Step S225).

FIG. 12A is a diagram schematically illustrating measurement information 720 transmitted to the measurement control section from the control device 300. The measurement information 720 includes a database key 721, a serial number 722, a sample rack ID 723, a sample number 724, a test tube position 725 and measuring item suction information 726. The database key 721 is information representing a key for extracting the information of the retrieving target from the measurement information management database 740. The serial number 722 is information representing the serial number which is applied to the sample rack 250 put on the transporting line 230 by the measurement control section 100. The sample rack ID 723 represents the information of the sample rack bar-code 251 acquired by the bar-code reader 111. The sample number 724 represents the information of the test tube bar-code 253 acquired by the bar-code reader 111. The test tube position 725 is information representing the position of the test tube 252 corresponding to the measurement information in the sample rack 250. The measuring item suction information 726 is information representing whether there are the orders of the measuring items and includes the information illustrated in FIG. 12B.

Measuring item suction information 727 illustrated in FIG. 12B includes a measuring item 728 and suction information 729. The measuring item 728 is information representing the measuring items which can be subjected to the measurement by the analyzer and the suction information 729 is information representing whether the measuring items are registered as the items to be subjected to the measurement or have been already suctioned for the measurement. 0 represents that the order for the measurement has been registered and the sample has not yet been suctioned. 1 represents that the order for the measurement has been registered and the suctioning has been completed. −1 represents that there is no order. For example, the measuring item suction information 727 illustrated in FIG. 12B represents that the order of the measuring item HBsAg has been registered and the suctioning of the sample has been completed, that the order of the measuring item HBsAb has not been registered, and that the order of the measuring item HCV has been registered and the suctioning of the sample has not yet been performed.

Returning to FIG. 6, when the measurement information is received, the measurement control section 100 stores the content of the measurement information (Step S314).

Next, when it is determined that the sample is present at the suction position 236 (Yes in Step S315) and that regarding the sample, there is an item to be subjected to the measurement in the order information 727 illustrated in FIG. 12B (Yes in Step S316), the measurement control section 100 starts the suctioning of the sample regarding the item (Step S317) and notifies the control device of that the sample suctioning is completed at a timing at which the suctioning of the sample is completed (Step S318). When the sample is not present at the suction position 236 (No in Step S315) or there is no measurement information about the sample at the suction position 236 (No in Step S316), the process proceeds to Step S319.

FIG. 13A is a diagram schematically illustrating a suction completion notification 730 transmitted to the control device 300 from the measurement control section 100 when the suctioning of the sample is completed. The suction completion notification 730 includes a database key 731 and measuring item suction information 732. The database key 731 represents the information for extracting the information of the retrieving target from the measurement information management database 740. The measuring item suction information 732 is information representing whether there are the orders of the measuring items and includes the information illustrated in FIG. 13B.

Measuring item suction information 735 illustrated in FIG. 13B includes a measurement item 736 and suction information 737. The measurement item 736 is information representing the measuring items which can be subjected to the measurement by the analyzer. The suction information 737 is information representing whether the measuring items are registered as the items to be subjected to the measurement or have been already suctioned for the measurement. 0 represents that the order for the measurement has been registered and the sample has not yet been suctioned. 1 represents that the order for the measurement has been registered and the suctioning has been completed. −1 represents that there is no order.

For example, the measuring item suction information 735 illustrated in FIG. 13B represents that the order of the measuring item HBsAg has been registered and the suctioning of the sample has been completed, that the order of the measuring item HBsAb has not been registered, and that the order of the measuring item HCV has been registered and the suctioning of the sample has not been completed.

Returning to FIG. 7, when the suction completion notification 730 of the sample illustrated in FIG. 13A is received from the measurement control section 100 (Yes in Step S230), the control device 300 searches the measurement information management database 740 on the basis of the database key 731 and updates the appropriate measuring item suction information 746 of the database key 741 by the received measuring item suction information 732 (Step S231).

Returning to FIG. 6, when it is determined that the sample rack 250 to be sent to the sample rack discharging section 242 is present on the transporting line 230 (Yes in Step S319), the measurement control section 100 performs the sending of the sample rack (Step S320). When the sensor 244 cannot detect that the sending lever 245 normally returns to the origin position thereof even after the sample rack 250 is sent by the sending lever 245 and a predetermined period of time passes, the measurement control section 100 performs an error processing to be described later.

Next, the measurement control section 100 updates the sample rack information management table 700 illustrated in FIG. 10 (Step S321). That is, when the sample rack is sent to the sample rack discharging section 242, the content stored in the data area of the second sample rack information 702 is copied to a first information storing area and the content of the second sample rack information 702 is erased. Then, when a new sample rack 250 is fed to the transporting line 230, the information acquired in Step S308 is stored in the data area of the second sample rack information. When the sensor 244 cannot detect that the sending lever 245 normally returns to the origin position thereof even after the sample rack 250 is sent by the sending lever 245 and a predetermined period of time passes, the measurement control section 100 performs the error processing to be described later.

In the measurement control section 100, when it is determined that the sample rack 250 to be sent to the sample rack discharging section 242 is not present on the transporting line 230 (No in Step S319), the process proceeds to Step S322.

Next, when it is determined that the overall measurement has been completed in the measurement control section 100 (Yes in Step S322), the state of the measurement control section 100 is set to a standby state and the control device 300 is notified of the state (Step S323). When the overall measurement has not been completed in the measurement control section 100, the process returns to Step S302.

Returning to FIG. 7, when the notification for analysis completion is received (Yes in Step S250), the control device 300 sets the measurement state to the standby state (Step S251).

When the user performs shutdown (Yes in Step S210), the measurement control section 100 is notified of a shutdown command from the control device 300 (Step S211). When the shutdown is not performed (No in Step S210), the process returns to Step S200.

Returning to FIG. 6, when the notification for shutdown is received from the control device 300 (Yes in Step S324), the measurement control section 100 performs the shutdown (Step S325), and after the completion of the shutdown, the measurement control section notifies the control device 300 of that the shutdown has been completed and turns the power off. When the notification for shutdown is not received (No in Step S324), the process returns to Step S324.

Returning to FIG. 7, when the notification for shutdown completion is received from the measurement control section 100 (Yes in Step S260), the control device 300 turns the power of the control device 300 off. When the notification for shutdown completion is not received from the measurement control section 100 (No in Step S260), the process returns to Step S220.

[Process upon Generation of Error]

When an error is generated in the transporting section 200 in the transverse sending of the rack (Step S303), the feeding of the rack (Step S305), the sending of the rack (Step S320) and the like in the flowchart illustrated in FIG. 6, the user of the immunological analyzer 1 has to reset the sample rack 250 present on the transporting line 230 in the sample rack setting section 221.

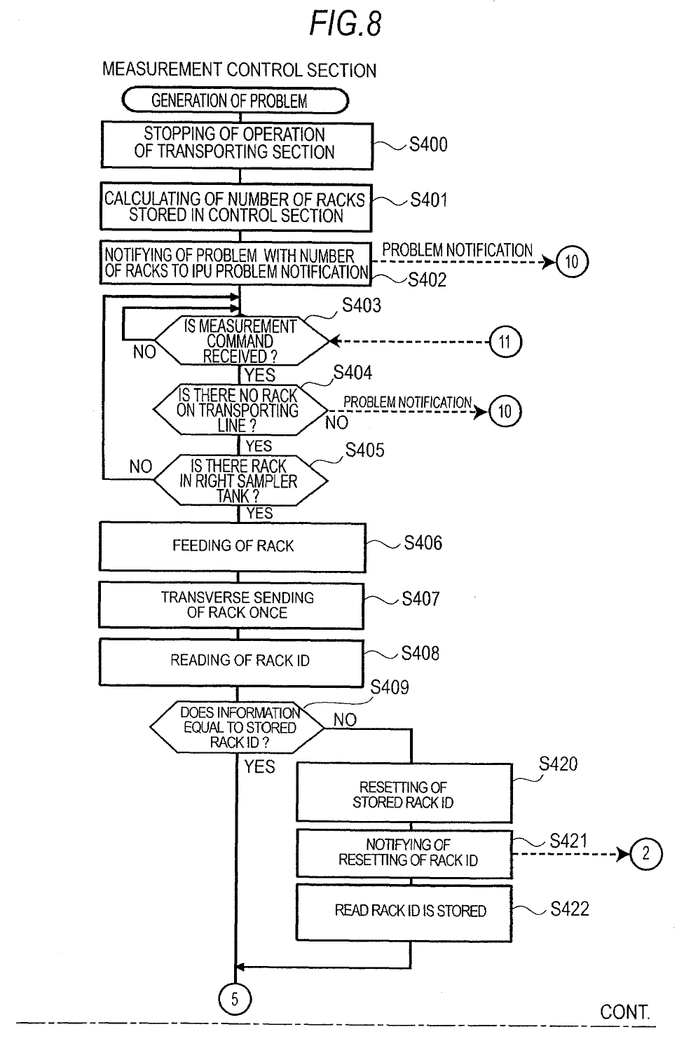

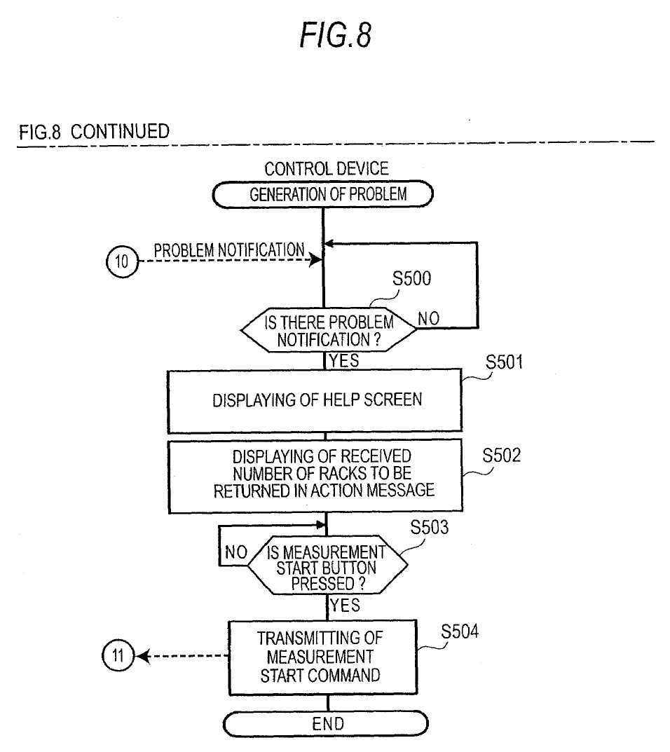

The process when an error is generated in the transporting section 200 during the measurement is shown in the flowchart of FIG. 8.

When the error of the transporting section 200 is detected, the measurement control section 100 stops the operation of the transporting section 200 (Step S400) and calculates the number of the sample racks 250 present on the transporting line 230 from the number of the information of the detected racks 250 in the sample rack information management table 700 illustrated in FIG. 10 (Step S401). Next, the measurement control section 100 notifies the control device 300 of the content of the error with the calculated number of the sample racks (Step S402).

FIG. 15 is a diagram schematically illustrating an error notification 800 transmitted to the control device 300 from the measurement control section 100. The error notification 800 includes an error No. 801 and a rack reset number 802. The error No. 801 is an ID uniquely corresponding to the error generated in the measurement control section 100. By using the ID as a key, the control device 300 can know the number of errors generated in the measurement control section 100 when a plurality of the errors are generated. The rack reset number 802 is information representing the number of the sample racks 250 on the transporting line 230 to be returned to the sample rack setting section 221 such that the user restarts the measurement.

For example, the error notification 800 illustrated in FIG. 15 represents that an error of which the error No. 801 is 377, that is, an error in the transverse sending of the rack is generated in the measurement control section 100 and the user is required to return one sample rack 250 on the transporting line 230 to the sample rack setting section 221 to restart the measurement.

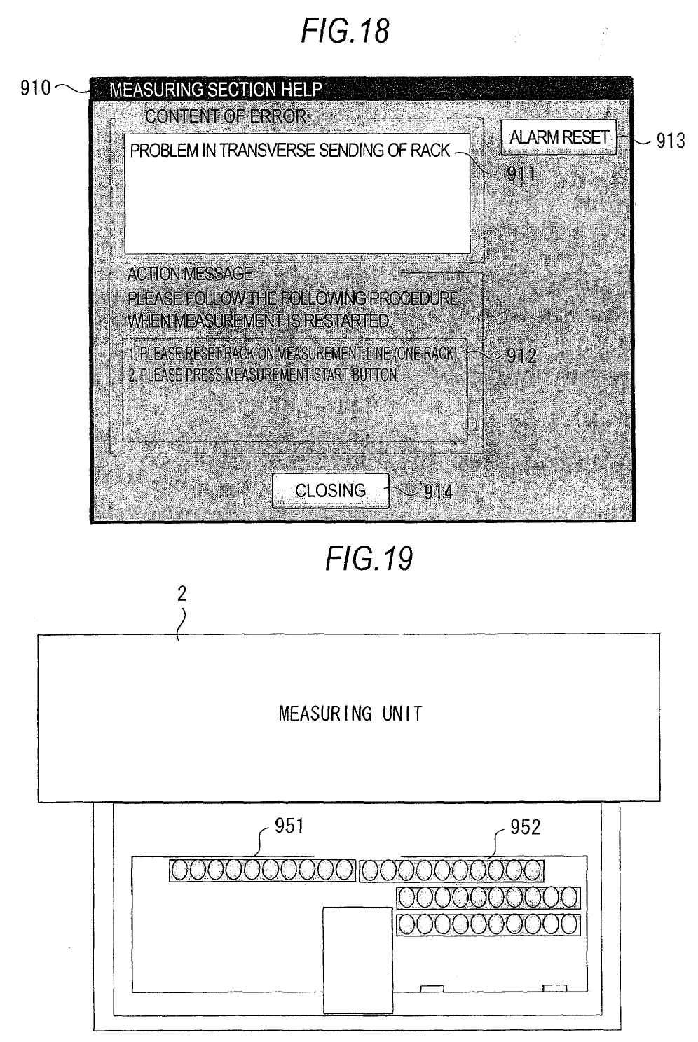

When an error notification 850 is received (Yes in Step S500), the control device 300 displays the help screen illustrated in FIG. 18 (Step S501) and displays the number of the sample racks to be returned in an action message (Step S502).

FIG. 18 is an example of a dialog displayed on the display 302 when the control device 300 is notified of the error from the measurement control section 100.

The help dialog 910 includes an error name 911, an action message 912, an alarm reset button 913 and a dialog closing button 914. The error name is information representing the name of the error detected by the measurement control section 100. The measurement control section 100 displays the error name corresponding to the information of the error No. 801 of the error notification 800 received from the control device 300. The action message 912 is information representing the operation procedure to be performed by the user to recover the error displayed in the error name 911. The alarm reset button 913 is a button for stopping an alarm ringing to notify the user of the error when the error is generated in the measurement control section 100. The dialog closing button 914 is a button for closing the help dialog 910.

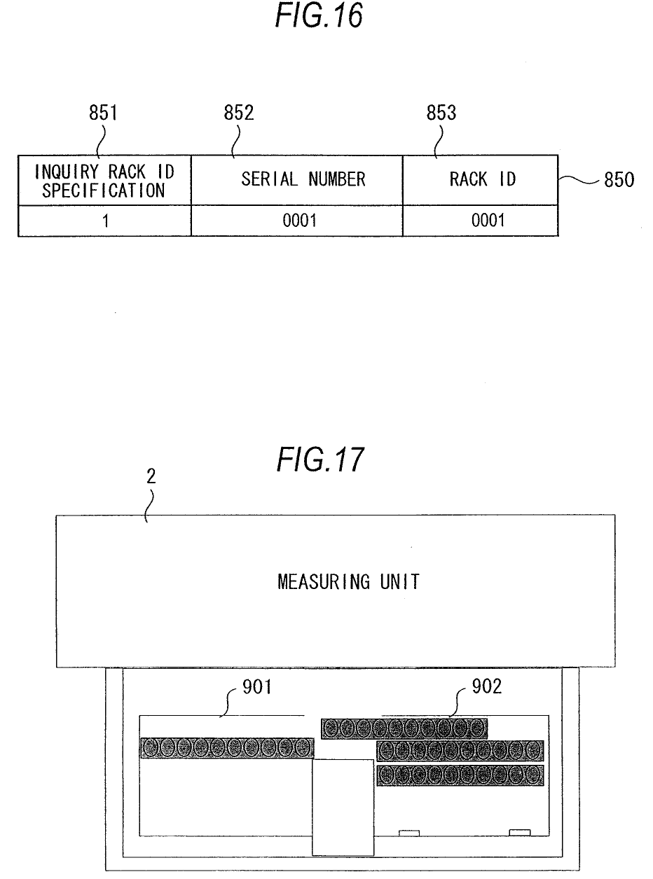

FIG. 17 is an example of the state of the sample rack 250 in the transporting section 200 upon display of the help dialog 910 illustrated in FIG. 18.

By resetting a sample rack 901 illustrated in FIG. 17 in front of a sample rack 902 (on the side of the measuring unit 2) and restarting the measurement, the user can restart the measurement by using the sample which has not yet been suctioned in the sample rack 901.

Next, when the user resets the sample rack 250 present on the transporting line 230 in the sample rack setting section 221 in accordance with the help dialog illustrated in FIG. 18 and presses the measurement start button 611 to restart the measurement (Yes in Step S503), the measurement control section 100 is notified of an instruction for measurement restart from the control device 300 (Step S504). When the measurement start button 611 is not pressed (No in Step S503), the process returns to Step S503.

When the instruction for measurement restart is received from the control device 300 (Yes in Step S403), the measurement control section 100 determines whether the sample rack 250 is present on the transporting line 230 on the basis of the output of the sensors 244 and 231 (Step S404). When the sample rack 250 does not exist on the transporting line 230 (Yes in Step S404), the process proceeds to Step S405 and determining whether the sample rack 250 is present in the sample rack setting section 221 is performed (Step S405). When it is determined that the sample rack 250 is present in the sample rack setting section 221 (Yes in Step S405), the sample rack 250 is fed to the transporting line 230 (Step S406), the sample rack 250 fed to the transporting line 230 is transversely sent by one pitch (Step S407) and the bar-code 251 adhered to the sample rack 250 is read (Step S408). When the sample rack 250 is present on the transporting line 230 (No in Step S404), the control device 300 is notified of the error.

Further, when the sample rack 250 is not present in the sample rack setting section 221 (No in Step S405), the process returns to Step S403.

Next, when there the information of the sample rack bar-code 251 acquired in Step S408 equals to the information in the sample rack information management table 700 stored in the measurement control section 100 (Yes in Step S409), the process proceeds to Step S309 illustrated in FIG. 6. When the information of the sample rack bar-code 251 acquired in Step S408 does not equal to the information in the sample rack information management table 700 (No in Step S409), the content of the sample rack information management table 700 is completely erased (Step S420) and a sample rack information nonavailability notification is transmitted to the control device 300 (Step S421). After that, the measurement control device 100 stores the information of the bar-code acquired by the bar-code reader 111 in the sample rack information management table 700 (Step S422) and the process proceeds to Step S309 illustrated in FIG. 6.

FIG. 16 is a diagram schematically illustrating a sample rack information nonavailability notification 850 transmitted to the control device 300 from the measurement control section 100. The sample rack information nonavailability notification 850 includes an inquiry key 851, a serial number 852 and a sample rack ID 853.

The inquiry key 851 is information representing whether the serial number 852 is used or the sample rack ID 853 is used as a key of the order inquiry to the control device 300 from the measurement control section 100. The serial number 852 is information representing the serial number applied to the sample rack 250 put on the transporting line 230 by the measurement control section 100. The sample rack ID 853 represents the information of the sample rack bar-code 251 acquired by the bar-code reader 111.

For example, the nonavailability notification 850 illustrated in FIG. 16 represents that the measurement information stored in the measurement information management database 740, of which the sample rack ID 853 is 0001, is excluded from the retrieving target when the measurement information inquiry data 710 illustrated in FIG. 11 is received.

When the sample rack information nonavailability notification 850 is received from the measurement control section 100 (Yes in Step S240), the control device 300 erases the appropriate measurement information from the retrieving target (Step S241) and applies a gray color as a background color to the registration state display box 604 in which the appropriate measurement information is registered (Step S242) As a result, the user can know that the appropriate measurement information about the sample rack is nonavailable.

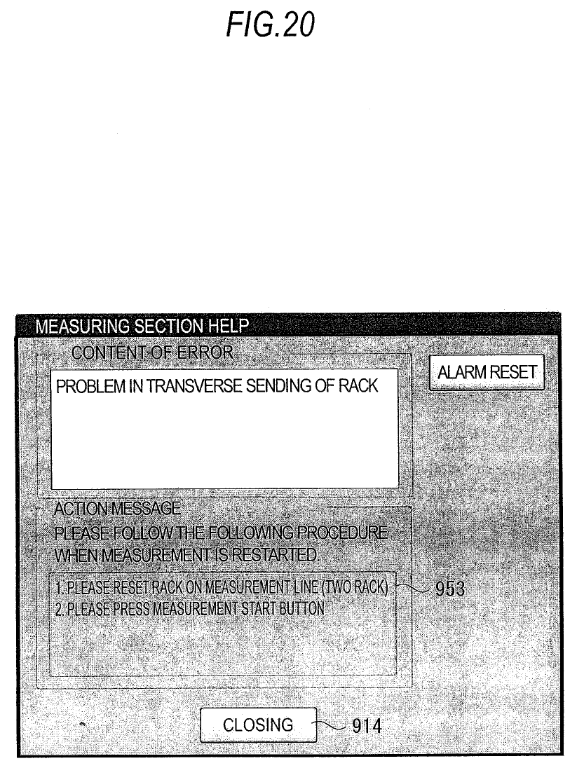

When a sample rack 951 and a sample rack 952 are present on the transporting line 230 as illustrated in FIG. 19 and an error is generated in the process of the transporting section 200 such as the transverse sending of the racks (Step S303) and the feeding of the racks (Step S305), the control device 300 displays the help screen illustrated in FIG. 20 on the display 302 of the control device 300.

The user recognizes an action message 953 and can know that two sample racks 250 on the transporting line 230 are required to be reset in the sample rack setting section 221 to recover the error.

In this first embodiment, although it has been described that the number of the sample racks 250 which can be accommodated on the transporting line 230 is two, the sample racks 250 which can be accommodated on the transporting line 230 may be three or more.

In this first embodiment, although it has been described that the control device 300 displays the number of the sample racks 250 returning to the sample rack setting section 221 from the transporting line 230 in the help dialog 910 when the error notification 850 is received from the measurement control section 100, the information of the sample rack bar-code 251 read by the bar-code reader 111 may be displayed.

In this first embodiment, although it has been described that the identification information of the sample rack bar-code 251 adhered to the sample rack 250 is acquired by using the bar-code reader 111, the serial number may be used without the use of the bar-code reader 111 to calculate the number of the sample racks 250 to be reset in the sample rack setting section 221, which are present on the transporting line 230.

In this first embodiment, although it has been described that the information of the sample rack 250 sent from the sample rack information management table 700 is erased at a timing at which the sample rack 250 present on the transporting line 230 is sent to the sample rack discharging section 242, the information of the sample rack 250 sent from the sample rack information management table 700 may be erased at a timing at which the measurement result is acquired. Accordingly, when the measurement result cannot be obtained due to the error generated in the immunological analyzer 1, the user can easily know which sample rack 250 is to be returned to the sample rack setting section 221 to restart the measurement.

In addition, the information of the sample rack 250 from the sample rack information management table 700 may be erased at a timing at which the suctioning of the samples required to be subjected to the measurement in all of the test tubes 252 held in the sample rack 250 is completed. Accordingly, when the error is generated in the immunological analyzer 1, the user can return only the sample rack 250 holding the test tube 252 in which the sample has not yet been suctioned to the sample rack setting section 221 and has no need to return the sample rack 250 holding the test tube 252 not required to suction the sample therein for the measurement to the sample rack setting section 221.

In this first embodiment, although it has been described that the error is detected in the transporting section 200, the information of the sample rack 250 to be reset in the sample rack setting section 221 may be provided by performing the error processing illustrated in FIG. 8 even when the error is detected in the measuring sections such as the suction section 105, urgent sample transporting section 101, reagent mounting section 106 and the reaction sections 107.

### Second Embodiment

Next, a second embodiment of the invention will be described in detail. Since the configurations of the measuring unit 2 and the control device 300 in the immunological analyzer 1 are the same in the first embodiment of the invention, the description thereof will be omitted.

[Overall Process]

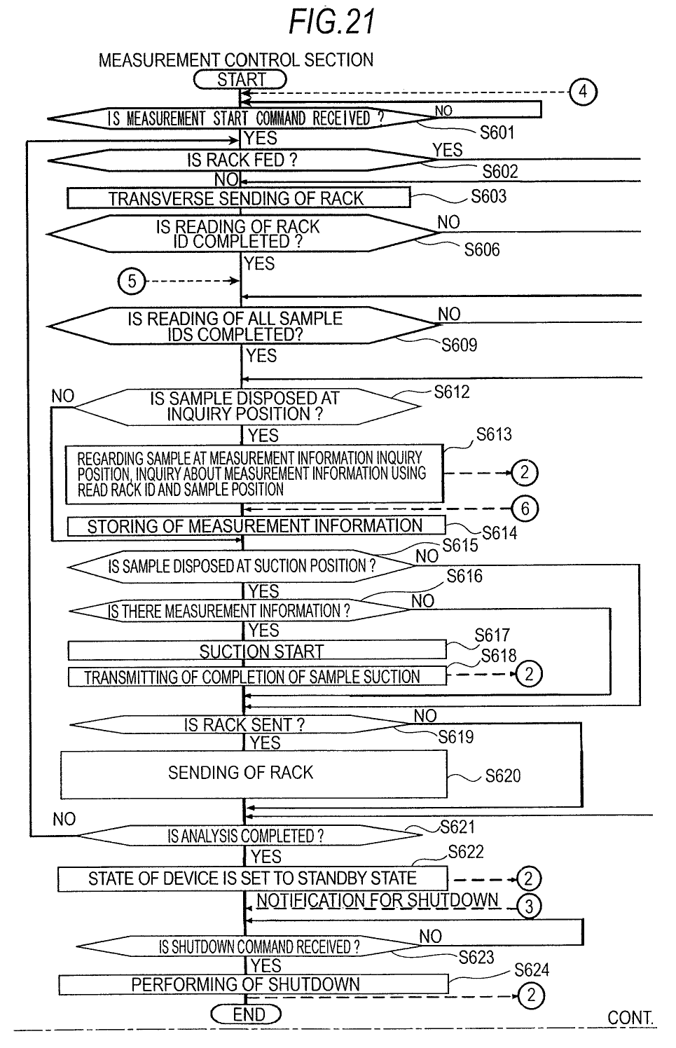

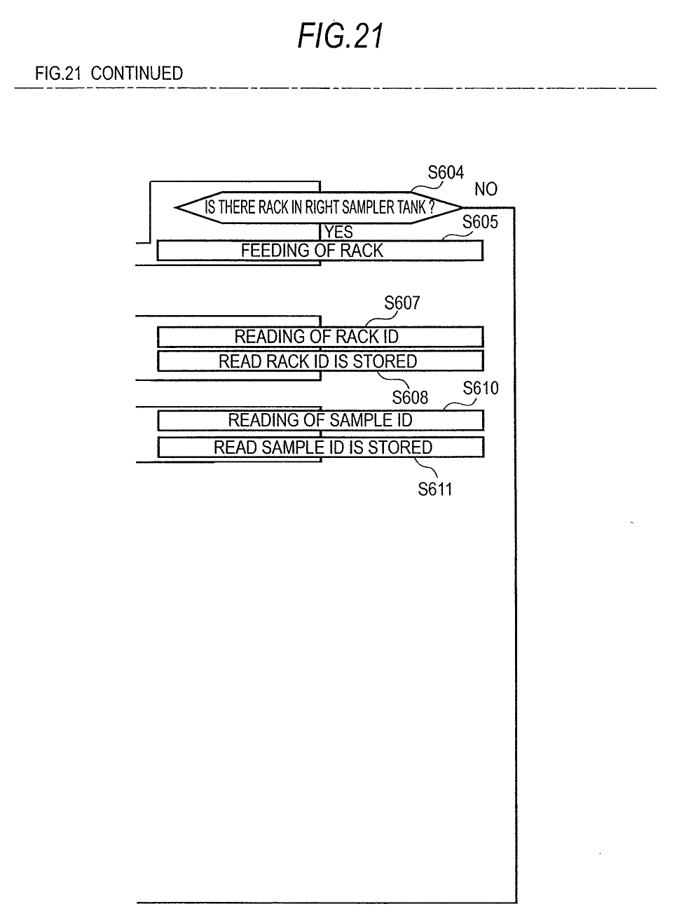

Using FIGS. 21 to 26, the sample measuring process performed by the immunological analyzer 1 according to the second embodiment will be described.

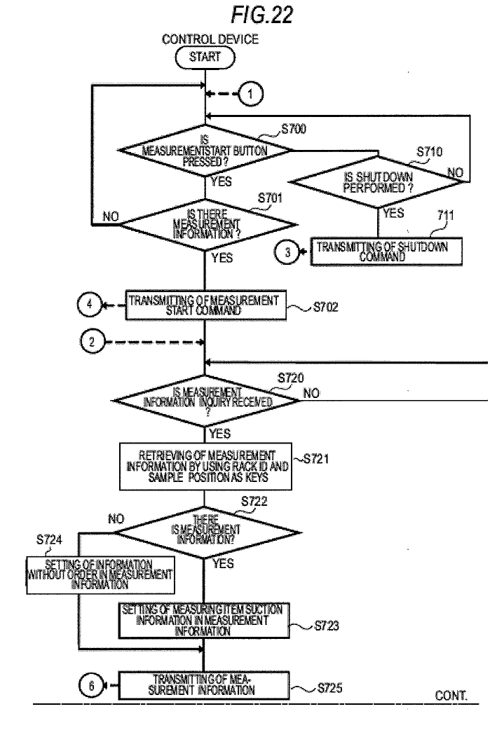

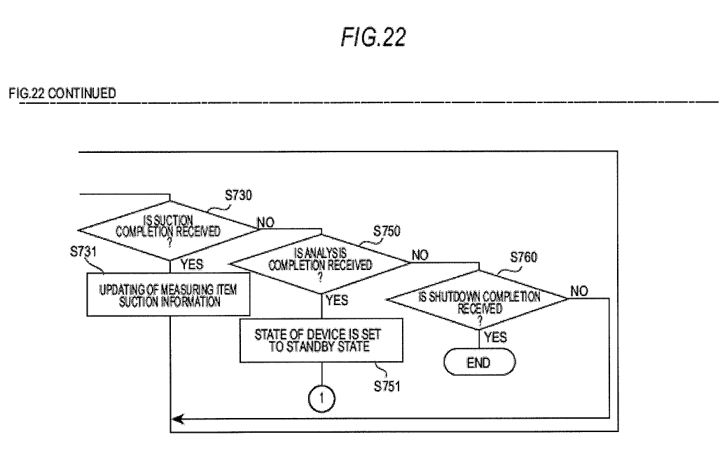

FIGS. 21 to 22 are flowcharts illustrating the processes when the measurement is normally performed in the immunological analyzer 1.

In Step S700 illustrated in FIG. 22, when the measurement start button 611 displayed on the display 302 of the control device 300 is pressed by a user (Yes in Step S700), determining whether measurement information is input by the user is performed (Step S701). When the measurement information is input by the user (Yes in Step S701), the measurement control section 100 is notified of measurement start (Step S702). When the measurement start button 611 is pressed by the user but the measurement information is not input (No in Step S701), the process returns to Step S700.

Moving to FIG. 21, when the notification for measurement start is received (Yes in Step S601), the measurement control section 100 determines whether the sample rack 250 is fed to the transporting line 230 (Step S602). When the notification for measurement start is not received (No in Step S601), the process returns to Step S601. In Step S602, when it is determined the sample rack 250 is to be fed (Yes in Step S602), determining whether the sample rack 250 is present in the sample rack setting section 221 is performed on the basis of the output of the sensor 226 (Step S604). When the sample rack 250 is present in the sample rack setting section 221 (Yes in Step S604), the sample rack 250 is fed to the transporting line 230 by the sample rack feeding mechanism section 222 (Step S605).

In this Step S605, when the sample rack arrival sensor 231 cannot detect that the sample rack 250 is fed even after the sample rack feeding mechanism section 222 is driven and a predetermined period of time passes, the measurement control section performs the process upon generation of the error to be described later.

Further, when returning of the sample rack feeding mechanism section 222 to the origin position thereof cannot be detected on the basis of the output of the sensor 225 even after the feeding of the sample rack 250 to the transporting line 230 is completed by the sample rack feeding mechanism section 222 and a predetermined period of time passes, the measurement control section 100 performs the process upon generation of the error to be described later.

When the sample rack 250 is not present in the sample rack setting section 221 (No in Step S604), the process proceeds to Step S621.

When it is determined that the sample rack 250 is not to be fed, that is, when a new sample rack 250 is fed to the transporting line 230 and it is determined that the new sample rack interferes with the sample rack 250 already present on the transporting line (No in Step S602), the measurement control section 100 does not perform the feeding of the sample rack 250 and the process proceeds to Step S603.

Next, the sample rack 250 on the transporting line 230 is transversely sent once (Step S603). In this Step S603, when it is detected on the basis of the output of the sensors 235a and 235b that the transverse sending levers 233 are not normally operated, or that the sample rack 250 is moved upon stopping of the motor 234, the measurement control section 100 performs the process upon generation of the error to be described later.

Next, the measurement control section 100 checks whether the information of the sample rack bar-code 251 has been acquired by the bar-code reader 111 (Step S606). When the information of the sample rack bar-code 251 has not yet been acquired (No in Step S606), the sample rack ID of the sample rack bar-code 251 is acquired by the bar-code reader 111 (Step S607) and the acquired rack ID of the sample rack bar-code 251 is stored (Step S608).

When the information of the sample rack bar-code 251 has been acquired (Yes in Step S606) or when the process proceeds to Step S609 after Step S608 and there is the test tube bar-code 253 of the test tube 252 held in the sample rack 250, which has not yet been acquired (No in Step S609), the sample ID of the test tube bar-code 253 which has not yet been acquired is acquired by the bar-code reader 111 (Step S610) and the acquired information is stored (Step S611).

Next, when the test tube 252 is present at the measurement information inquiry position 237 (Yes in Step S612), an inquiry is made to the control device 300 about the measurement information of the test tube 252 present at the measurement information inquiry position 237 by using the rack ID of the sample rack bar-code 251 and the test tube position in the sample rack 250 holding the test tube 252 as keys (Step S613). When the test tube 252 is not present at the measurement information inquiry position 237 (No in Step S612), the process proceeds to Step S615.

Returning to FIG. 22, when the measurement information inquiry data 710 illustrated in FIG. 11 is received (Yes in Step S720), the control device 300 retrieves the measurement information from the measurement information stored in the predetermined area of the hard disk 313 illustrated in FIG. 2 by using the rack ID of the sample rack bar-code 251 and the test tube position in the sample rack 250 holding the test tube 252 as keys (Step S721).

FIG. 11 is the diagram schematically illustrating the measurement information inquiry data 710 transmitted to the control device 300 from the measurement control section 100. Since the measurement information inquiry data 710 has been described in detail in the first embodiment, the description thereof will be omitted.

FIG. 9 is the example of the measurement information registering screen where the user inputs the measurement information. Since the measurement information registering screen has been described in detail in the first embodiment, the description thereof will be omitted.

FIG. 14A is the diagram schematically illustrating the measurement information management database 740 for managing the measurement information stored in the hard disk 313. Since the measurement information management database 740 has been described in detail in the first embodiment, the description thereof will be omitted.

Returning to FIG. 22, when there is the appropriate measurement information after the retrieval of the measurement information (Yes in Step 722), the database key 741 and the measuring item suction information 746 are added to the measurement information inquiry data 710 (Step S723) and the measurement control section 100 is notified of the order information illustrated in FIG. 11 (Step S725). When there is not the appropriate measurement information after the retrieval of the measurement information (No in Step S722), information without an order is added to the measurement information inquiry data 710 and the measurement control section 100 is notified (Step S725).

FIG. 12A is the diagram schematically illustrating the measurement information 720 transmitted to the measurement control section from the control device 300. Since the measurement information 720 has been described in detail in the first embodiment, the description thereof will be omitted.

Returning to FIG. 21, when the measurement information is received, the measurement control section 100 stores the content of the measurement information (Step S614).

Next, when it is determined that the sample is present at the suction position 236 (Yes in Step S615) and that regarding the sample, there is an item to be subjected to the measurement in the order information 727 illustrated in FIG. 12B (Yes in Step S616), the measurement control section 100 starts the suctioning of the sample regarding the item (Step S617) and notifies the control device of that the sample suctioning is completed at a timing at which the suctioning of the sample is completed (Step S618). When the sample is not present at the suction position 236 (No in Step S615) or there is no measurement information about the sample at the suction position 236 (No in Step S616), the process proceeds to Step S619.

FIG. 13A is the diagram schematically illustrating the suction completion notification 730 transmitted to the control device 300 from the measurement control section 100 when the suctioning of the sample is completed. Since the suction completion notification 730 has been described in detail in the first embodiment, the description thereof will be omitted.

Returning to FIG. 22, when the suction completion notification 730 of the sample illustrated in FIG. 13A is received from the measurement control section 100 (Yes in Step S730), the control device 300 searches the measurement information management database 740 on the basis of the database key 731 and updates the appropriate measuring item suction information 746 of the database key 741 by the received measuring item suction information 732 (Step S731).

Returning to FIG. 21, when it is determined that the sample rack 250 to be sent to the sample rack discharging section 242 is present on the transporting line 230 (Yes in Step S619), the measurement control section 100 performs the sending of the sample rack (Step S620). When the sensor 244 cannot detect that the sending lever 245 normally returns to the origin position thereof even after the sample rack 250 is sent by the sending lever 245 and a predetermined period of time passes, the measurement control section 100 performs an error processing to be described later.

When the sensor 244 cannot detect that the sending lever 245 normally returns to the origin position thereof even after the sample rack 250 is sent by the sending lever 245 and a predetermined period of time passes, the measurement control section 100 performs the error processing to be described later.

In the measurement control section 100, when it is determined that the sample rack 250 to be sent to the sample rack discharging section 242 is not present on the transporting line 230 (No in Step S619), the process proceeds to Step S622.

Next, when it is determined that the overall measurement has been completed in the measurement control section 100 (Yes in Step S621), the state of the measurement control section 100 is set to a standby state and the control device 300 is notified of the state (Step S622). When the overall measurement has not been completed in the measurement control section 100, the process returns to Step S602.

Returning to FIG. 22, when the notification for analysis completion is received (Yes in Step S750), the control device 300 sets the measurement state to the standby state (Step 751).

When the user performs shutdown (Yes in Step S710), the measurement control section 100 is notified of a shutdown command from the control device 300 (Step S711). When the shutdown is not performed (No in Step S710), the process returns to Step S700.

Returning to FIG. 21, when the notification for shutdown is received from the control device 300 (Yes in Step S623), the measurement control section 100 performs the shutdown (Step S624), and after the completion of the shutdown, the measurement control section notifies the control device 300 of that the shutdown has been completed and turns the power off. When the notification for shutdown is not received (No in Step S623), the process returns to Step S623.

Returning to FIG. 22, when the notification for shutdown completion is received from the measurement control section 100 (Yes in Step S760), the control device 300 turns the power of the control device 300 off. When the notification for shutdown completion is not received from the measurement control section 100 (No in Step S760), the process returns to Step S720.

[Process upon Generation of Error]

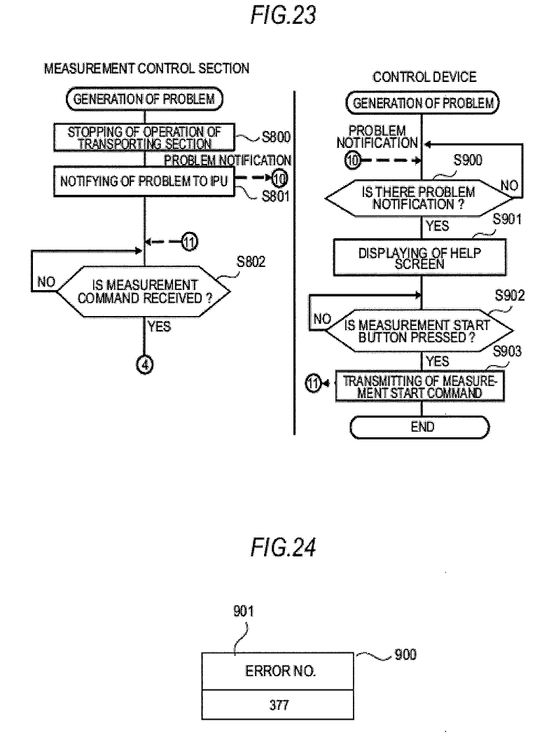

The flowchart of FIG. 23 illustrates processes for the case where an error is generated in the transporting section 200 in the transverse sending of the rack (Step S603), the feeding of the rack (Step S605), the sending of the rack (Step S620) and the like in the flowchart illustrated in FIG. 21.

When the error of the transporting section 200 is detected, the measurement control section 100 stops the operation of the transporting section 200 (Step S800) and notifies the control device 300 of the content of the error (Step S801).

FIG. 24 is a diagram schematically illustrating an error notification 900 transmitted to the control device 300 from the measurement control section 100. The error notification 900 includes an error No. 901. The error No. 901 is an ID uniquely corresponding to the error generated in the measurement control section 100. By using the ID as a key, the control device 300 can know the number of errors generated in the measurement control section 100 when a plurality of the errors are generated.

For example, the error notification 900 illustrated in FIG. 24 represents that an error of which the error No. 901 is 377, that is, an error in the transverse sending of the rack is generated in the measurement control section 100.

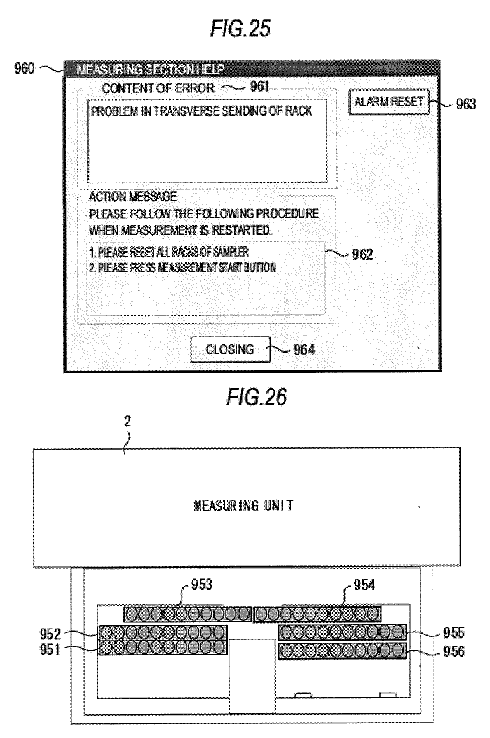

Returning to FIG. 23, when the error notification 850 is received (Yes in Step S900), the control device 300 displays the help screen illustrated in FIG. 25 and displays on the help screen the action message representing that all of the racks are to be returned to predetermined positions (Step S901).

FIG. 25 is an example of the help screen displayed on the display 302 by the control device 300 when the error notification is received from the measurement control section 100.

A help screen 960 includes an error name 961, an action message 962, an alarm reset button 963 and a screen closing button 964. The error name 961 is information representing the name of the error detected by the measurement control section 100. The measurement control section 100 displays the error name corresponding to the information of the error No. 801 of the error notification 800 received from the control device 300. The action message 962 is information representing the operation procedure to be performed by the user to recover the error displayed in the error name 961. The alarm reset button 963 is a button for stopping an alarm ringing to notify the user of the error when the error is generated in the measurement control section 100. The screen closing button 964 is a button for closing the help screen 960.

FIG. 26 is an example of the state of the sample racks 250 in the transporting section 200 when the help screen 960 illustrated in FIG. 25 is displayed.

The user resets sample racks 951 to 956 illustrated in FIG. 26, which are present on the transporting line 230 and in the sample rack setting section 221 and the sample rack discharging section 242, in the sample rack setting section 221 and restarts the measurement to restart the stopped measurement. At this time, the sample racks 250 to be set in the sample rack setting section 221 may be set in a random sequence. For example, the sample racks 951 to 954 may be se t after a sample rack 906 is set.

Returning to FIG. 23, when the user resets the sample racks 250, which are present on the transporting line 230 and in the sample rack discharging section 242, in the sample rack setting section 221 in accordance to the help screen illustrated in FIG. 25 and presses the measurement start button 611 to restart the measurement (Yes in Step S902), the measurement control section 100 is notified of an instruction for measurement restart from the control device 300 (Step S903). When the measurement start button 611 is not pressed (No in Step S902), the process returns to Step S902.

When the instruction for measurement restart is received from the control device 300 (Yes in Step S403), the measurement control section 100 allows the process to proceed to Step S602 illustrated in FIG. 21.

On the basis of the measurement information 720 which is received from the control device 300 and stored (Steps S614 to S616), the measurement control section 100 determines whether the sample at the suction position 236 is required to be suctioned. When it is determined that the sample is required to be suctioned, the measurement control section 100 controls the suction section 105 to suction the sample from the test tube 252 at the suction position 236. When it is determined that the sample is not required to be suctioned, the measurement control section 100 controls the sample rack transverse sending mechanism 232 to transversely send the sample rack 250.

In this second embodiment, although it has been described that the inquiry about the measurement information is made by using the rack ID of the sample rack bar-code 251 adhered to the sample rack 250 acquired by the bar-code reader 111 and the test tube position in the sample rack 250 holding the test tube 252 as keys, the inquiry about the measurement information may be made by using the sample ID of the test tube bar-code 253 acquired by the bar-code reader 111 as a key.

In this second embodiment, although it has been described that the user may reset the sample racks 951 to 956 illustrated in FIG. 26, which are present on the transporting line 230 and in the sample rack setting section 221 and the sample rack discharging section 242, in the sample rack setting section 221 in a random sequence when the error is generated in the transporting section 200, the sample racks may be reset in the sample rack setting section 221 in a sequence in which the sample racks is fed to the transporting line 230. For example, in FIG. 26, the sample racks 250 are set in the sample rack setting section 221 in a sequence of 951 to 956.

In this second embodiment, although it has been described that the CPU 310 of the control device 300 which controls the display 302 displaying the help screen is different from the measurement control section 100 which controls the suction section 105 and the transporting section 200, the display 302 for displaying the help screen may be included in the measuring unit 2 such that the measurement control section 100 controls the display.

In this second embodiment, although it has been described that one of −1 (without order), 0 (the order for the measurement is registered and the suction of the sample is not performed) and 1 (the order for the measurement is registered and the suction of the sample is completed) is set as the value which is set in the suction information 752 of the measuring item suction information 750, the information representing results in which the analysis of the sample is normally completed and in which the analysis is completed but the error is shown may be set. Accordingly, the user can easily subject the sample having the result in which the error is shown to remeasurement.

In this second embodiment, although it has been described that the error is detected in the transporting section 200, the information of the sample rack 250 to be reset in the sample rack setting section 221 maybe provided by performing the error processing illustrated in FIG. 23 even when the error is detected in the measuring sections such as the suctioning section 105, urgent sample transporting section 101, reagent mounting section 106 and the reaction sections 107.

What is claimed is:

1. An analyzer comprising:
a measuring section for measuring samples;
a transporting section for transporting a sample rack which holds sample container containing the sample to the measuring section;
a motion controller for controlling the measuring section and the transporting section;
an error detector for detecting an error of the analyzer;
a display section;
a display controller for displaying on the display section information representing handling of the sample rack present on the transporting section when the error detector detects the error; and
a restart command receiver for receiving an instruction for measurement restart when the error occurs in the analyzer, wherein the motion controller controls the measuring section and the transporting section so as to selectively suction a sample required to be suctioned when the restart command receiver receives the instruction for measurement restart.

2. The analyzer according to claim 1, wherein the transporting section includes a transporting line for transporting the sample rack to the measuring section, the analyzer further comprises a sample rack identification acquirer for acquiring information of the sample rack present on the transporting line and a memory for sample rack identification for storing the information of the sample rack acquired by the sample rack identification acquirer,
and the display controller displays the information representing handling of the sample rack present on the transporting section on the basis of the information of the sample rack stored in the memory for sample rack identification when the error detector detects the error of the analyzer.

3. The analyzer according to claim 2,
wherein the sample rack identification acquirer acquires identification information of the sample rack present on the transporting line.

4. The analyzer according to claim 2,
wherein the memory for sample rack identification stores the information of the sample rack when the sample rack identification acquirer acquires the information of the sample rack.

5. The analyzer according to claim 2,
wherein the error detector detects at least one error of the measuring section and the transporting section.

6. The analyzer according to claim 2, further comprising:
a sample rack discharge detector for detecting that the sample rack is discharged from the transporting line; and
a sample rack information eraser for erasing the information of the sample rack stored in the memory for sample rack identification,
wherein the sample rack information eraser erases the information of the sample rack discharged from the transporting line from the memory when the sample rack discharge detector detects that the sample rack is discharged from the transporting line.

7. The analyzer according to claim 2,
wherein the display controller calculates the number of sample rack present on the transporting line on the basis of the information of the sample rack stored in the memory for sample rack identification when the error detector detects the error, and displays on the display section information including the calculated number of sample rack as the information representing the handling of the sample rack present on the transporting section.

8. The analyzer according to claim 7,
wherein the information representing the handling of the sample rack is information representing the number of the sample rack present on the transporting line, which is to be returned to predetermined positions.

9. The analyzer according to claim 3,
wherein the memory for sample rack identification stores the identification information of the sample rack acquired by the sample rack identification acquirer, and the display controller extracts the identification information of the sample rack stored in the memory for sample rack identification when the error detector detects the error, and displays on the display section information including the extracted identification information of the sample rack as the information representing the handling of the sample rack present on the transporting section.

10. The analyzer according to claim 1,
wherein the transporting section includes a setting section for setting a sample rack which holds sample container, a storing section for storing the sample rack which holds a sample container in which a sample is suctioned by the measuring section and a transporting mechanism for transporting the sample rack to the storing section from the setting section, and the display controller displays on the display section information representing that all of sample racks present on the transporting section are set in the setting section as the information representing the handling of the sample rack when the error is generated in the analyzer.

11. The analyzer according to claim 1, further comprising:
an identification reader for acquiring identification information for identifying the sample rack or the sample container; and
a memory for measure progress for storing progress information of the measurement of the sample transported by the transporting section,
wherein the motion controller controls the measuring section and the transporting section to selectively suction a sample required to be suctioned by the measuring section on the basis of the identification information acquired by the sample rack identification acquirer and the progress information of the measurement stored in the memory for measure progress.

12. The analyzer according to claim 11,
wherein the memory for measure progress stores the progress information and the identification information of the sample in association with each other, or stores the progress information and the identification information of the sample rack and position of the sample in the sample rack in association with each other, and
wherein the motion controller controls the measuring section and the transporting section to selectively suction a sample required to be suctioned by the measuring section on the basis of the progress information stored in the memory for measure progress and the identification information acquired by the identification reader.

13. The analyzer according to claim 11,
wherein the progress information includes sample suction information representing whether the sample is suctioned, and the motion controller determines whether the sample is required to be suctioned by the measuring section on the basis of the sample suction information.

14. A measurement restarting method comprising:
transporting a sample rack to a measuring section by a transporting section for transporting the sample rack which holds sample container containing sample to the measuring section;
subjecting a sample to measurement by the measuring section for performing the measurement of the sample;
detecting an error of the analyzer;
displaying information representing handling of the sample rack present on the transporting section when the error of the analyzer is detected; and
selectively suctioning a sample required to be suctioned when an instruction for measurement restart is received after detection of the error.

15. The measurement restarting method according to claim 14,
wherein the transporting section includes a transporting line for transporting the sample rack to the measuring section, the analyzer acquires information of the sample rack present on the transporting line and stores the acquired information of the sample rack, and information representing handling of the sample rack present on the transporting section is displayed on the basis of the stored information of the sample rack when the error of the analyzer is detected.

16. The measurement restarting method according to claim 15,
wherein identification information of the sample rack present on the transporting line is acquired.

17. The measurement restarting method according to claim 15,
wherein the information of the sample rack is stored when identification information is acquired.

18. The measurement restarting method according to claim 15,
wherein at least one error of the measuring section and the transporting section is detected.

19. The measurement restarting method according to claim 15,
wherein it is detected that the sample rack is discharged from the transporting line, and the information of the sample rack discharged from the transporting line is erased when it is detected that the sample rack is discharged from the transporting line.

20. The measurement restarting method according to claim 15,
wherein the number of sample rack present on the transporting line is calculated on the basis of identification information of the sample rack present on the transporting line when the error of the analyzer is detected, and information including the number of the sample rack is displayed.
